(19) United States
(12) Patent Application Publication
(10) Pub. No.: US 2007/0254037 A1
(43) Pub. Date: Nov. 1, 2007

(54) Methods and Compositions for the Treatment of Cell Proliferation

(76) Inventor: Youri Popowski, Geneva (CH)

Correspondence Address: KNOBBE MARTENS OLSON & BEAR LLP, 2040 MAIN STREET, FOURTEENTH FLOOR, IRVINE, CA, 92614, US

(21) Appl. No.: 11/661,572

(22) PCT Filed: Aug. 30, 2005
(86) PCT No.: PCT/EP05/09324

§ 371 Date: Feb. 28, 2007

Related U.S. Application Data
(60) Provisional application No. 60/635,778, filed on Dec. 15, 2004.

(30) Foreign Application Priority Data
Dec. 15, 2004 (EP) 04447279.3

(51) Int. Cl. A61K 31/19; A61K 31/04; A61K 31/352; A61K 31/70; A61K 9/50; A61P 13/08; A61P 35/00

(52) U.S. Cl. 424/490; 514/23; 514/455; 514/557; 514/728

(57) ABSTRACT

The use of compositions which include one or more inhibitors of the citric acid, TCA, cycle and/or one or more inhibitors of oxidative phosphorylation for the treatment of cellular proliferation is disclosed. The composition is administered to or in the region of the proliferating cells or in a resection scar or cavity.

### BACKGROUND TO THE INVENTION

The treatment of conditions relating to cellular proliferation, malignant and benign, such as tumours, hyperproliferative scars, cheloid scars, and restenotic processes at the level of a duct have several disadvantages, such as, for example, high toxicity, low efficacy, expense and the requirement for repeated or continuous administration.

The use of metabolic pathway inhibitors for the treatment cellular proliferation is known in the prior art. For example, US 2003/30181393 describes inhibitors of glycolysis and oxidative phosphorylation; US 2003/0087961 described the use of inhibitors of glycolysis; EP 1372646, WO 02/072077, WO 2004/024676 described the use of glycolysis and transaminase inhibitors; US2002/0187534 and US2002/0024050 describe the blocking of fatty acid synthase to inhibit cellular proliferation. Regarding specific pathway inhibitors, Arcadi, Journal of Urology, 160, 2402-2406 (1998) describes the administration of rhodamine for the treatment of prostate cancer; Modica-Napitano et al, Biochemical and Biophysical Research Communications 118 (3), 717-723 (1984) and Modica-Napitano et al, Cancer Research 47, 4361-4365 (1987) describe the in vitro effect of rhodamine on oxidative phosphorylation; Juang, Molecular Genetics and Metabolism 81, 244-252 (2004) describes inhibition of the aconitase gene by antisense therapy; Lauble et al, Proc. Natl. Acad, Sci, USA 93, 13699-13703 (1996) describes the crystal structure of the enzyme inhibitor complex of fluorocitrate and aconitase.

The present invention aims to overcome the problems of the prior art by providing alternative an improved treatments for cellular proliferation.

### SUMMARY OF SOME EMBODIMENTS OF THE INVENTION

One embodiment of the present invention is a use of a composition comprising one or more inhibitors of the citric acid, TCA, cycle and/or one or more inhibitors of oxidative phosphorylation for the manufacture of a medicament for the treatment of cellular proliferation, wherein said composition is directly administered to or in the region of the proliferating cells.

One embodiment of the present invention is a use of a composition comprising one or more inhibitors of the citric acid, TCA, cycle, one or more slow release agents and optionally one or more inhibitors of oxidative phosphorylation for the manufacture of a medicament for the treatment of cellular proliferation, wherein said composition is administered into the proliferating cell mass.

One embodiment of the present invention is a use of a composition comprising one or more inhibitors of the citric acid, TCA, cycle, one or more slow release agents and optionally one or more inhibitors of oxidative phosphorylation for the manufacture of a medicament for sensitising a cellular proliferation to treatment by radiotherapy, wherein said composition is administered into the proliferating cell mass prior to radiotherapy.

One embodiment of the present invention is a use of a composition comprising one or more inhibitors of the citric acid, TCA, cycle, one or more slow release agents and optionally one or more inhibitors of oxidative phosphorylation for the manufacture of a medicament for sensitising a cellular proliferation to treatment by chemotherapy, wherein said composition is administered into the proliferating cell mass prior to chemotherapy.

One embodiment of the present invention is a use as described above, wherein said TCA cycle and oxidative phosphorylation inhibitors are administered separately, simultaneously or sequentially.

Another embodiment of the present invention is a use as described above, wherein administration leads to inhibition of the TCA cycle and oxidative phosphorylation pathway.

Another embodiment of the present invention is a use as described above, wherein said TCA cycle inhibitor is an inhibitor of one or more of pyruvate dehydrogenase, citrate synthase, aconitase, isocitrate lyase, alpha-ketoglutarate dehydrogenase complex, succinyl CoA synthetase, succinate dehydrogenase, fumarase, malate synthase, glutaminase and pyruvate dehydrogenase complex.

One embodiment of the present invention is a use as described above, wherein said TCA cycle inhibitor is any of arsenite, hypoglycin A, methylenecyclopropylacetic acid, alloxan, PNU, p-benzoquinone, fluoroacetate, halogenated acetates (iodo-, bromo-, chloro-acetate), halogenated acetyl-CoA (fluoroacetyl-CoA, bromoacetyl-CoA, chloroacetyl-CoA, iodoacetyl-CoA), halogenated crotonate (fluoro-, iodo-, bromo-, chloro-crotonate), halogenated ketone bodies, (chloro-, fluoro-, bromo-, iodoaceto-acetate, fluoro-, chloro-, bromo-, iodo-butyrate, fluoro-, chloro-, bromo-, iodo-acetone), halogenated oleate (iodo, bromo, chloro, fluoro-oleate), halogenated citrate, halogenated citrate 2R, 3R isomer (fluoro-, bromo-, chloro-, iodo-citrate), dichlorovinyl-cysteine, halogenated aminoacids, malonate, pentachlorobutadienyl-cysteine, 2-bromohydroquinone, 3-nitropropionic acid, cis-crotonalide fungicides, glu-hydroxyoxamate, p-chloromercuriphenylsulphonic acid, L-glutamate gamma-hydroxamate, p-chloromercuriphenylsulphonic acid, acivicin (alpha-amino-3-chloro-4,5-dihydro-5-isoxazoleacetic acid, halogenated glutamine (fluoro, iodo, chloro, bromo-glutamine), or halogenated glutamate (fluoro, iodo, chloro, bromo-glutamate), a stereoisomer, tautomer, racemate, prodrugs, metabolite thereof, or a pharmaceutically acceptable salt, base, ester or solvate thereof.

One embodiment of the present invention is a use as described above, wherein said TCA cycle inhibitor is a compound of formula (I) below or a stereoisomer, tautomer, racemate, prodrug, metabolite thereof, or a pharmaceutically acceptable salt, base, ester or solvate thereof, where X is halide, a sulfonate, a carboxylate, an alkoxide, an amine oxide or OH.

One embodiment of the present invention is a use as described above, where in formula (I):
a halide is selected from the group consisting of: fluoride, bromide, chloride, and iodide,
a sulfonate may be selected from the group consisting of: triflate, mesylate and tosylate,
a carboxylate may be selected from the group consisting of: methoxylate and ethyloxylate,
an alkoxide may be selected from the group consisting of: methoxide and ethoxide,
an amine oxide is dimethylamine oxide, and
where the stereochemistry is 2R, 3R.

One embodiment of the present invention is a use as described above wherein said TCA cycle inhibitor is a compound of formula (II) below or a stereoisomer, tautomer, racemate, prodrug, metabolite thereof, or a pharmaceutically acceptable salt, base, ester or solvate thereof where X is a halide, a sulfonate, a carboxylate, an alkoxide, an amine oxide, or an OH.

One embodiment of the present invention is a use as described above, where in formula (II):
the halide is selected from the group consisting of: fluoride, bromide, chloride, and iodide,
the sulfonate is selected from the group consisting of: triflate, mesylate and tosylate,
the carboxylate is selected from the group consisting of: methoxylate and ethyloxylate,
the alkoxide is selected from the group consisting of: methoxide and ethoxide, and
the amine oxide is dimethylamine oxide.

Another embodiment of the present invention is a use as described above, wherein said TCA cycle inhibitor is any of p-benzoquinone, thiaminase, fluoroacetamide, halogenated ketone bodies, chloroacetoacetate, fluoroacetoacetate, fluorohydroxybutyrate, chlorohydroxybutyrate, bromohydroxybutyrate), halogenated acetic acid, chloracetic acid, 6-diazo-5-oxo-L-norleucine (DON) or a stereoisomer, tautomer, racemate, prodrug, metabolite thereof, or a pharmaceutically acceptable salt, base, ester or solvate thereof.

Another embodiment of the present invention is a use as described above, wherein said TCA cycle inhibitor is any of fluoroacetate, arsenite, acetoacetate, and betahydroxy butyrate or a stereoisomer, tautomer, racemate, prodrug, metabolite thereof, or a pharmaceutically acceptable salt, base, ester or solvate thereof.

Another embodiment of the present invention is a use as described above, wherein said TCA cycle inhibitor, when comprising a halogen atom, comprises a halogen radioisotope in place of a stable halogen atom.

One embodiment of the present invention is a use as described above, wherein said oxidative phosphorylation inhibitor is an inhibitor of one or more of complex I (NADH coenzyme Q reductase), II (succinate-coenzyme Q reductase), III (coenzyme Q cytochrome C reductase), IV (cytochrome oxydase), and V (F0-F1, ATP synthase).

One embodiment of the present invention is a use as described above wherein said oxidative phosphorylation inhibitor is any of rotenone, amytal, 1-methyl-4-phenylpyridinium, paraquat, myxothiazol, antimycin A, ubisemiquinone, cytochrome C, 4,6-diaminotriazine derivatives, cyanide, hydrogen sulfide, azide, formate, phosphine, carbon monoxide, 4′-demethyl-epipodophyllotoxin thenylidene glucoside, tritylthioalanine, carminomycin, piperazinedione, dinitrophenol, dinitrocresol, 2-hydroxy-3-alkyl-1,4-naphtoquinones, apoptolidin aglycone, oligomycin, ossamycin, clofazimine cytovaricin, naphtoquinone derivatives, dichloroallyl-lawsone, lapachol, rhodamine, rhodamine 123, rhodamine 6G, carbonyl cyanide p-trifluoromethoxyphenylhydrazone, cyhexatin, dichlorodiphenyltrichloroethane (DDT), chlordecone, arsenate, pentachlorophenol, benzonitrile, thiadiazole herbicides, salicylate, cationic amphilic drugs, amiodarone, perhexiline, gramicidin, calcimycin, pentachlorobutadienyl-cysteine, trifluorocarbonylcyanide phenylhydrazone, atractyloside, lysophospholipids, n-ethylmaleimide, mersanyl, or p-benzoquinone.

Another embodiment of the present invention is a use as described above, wherein said inhibitor of oxidative phosphorylation is an inhibitor of enzyme complex I (NADH coenzyme Q reductase).

Another embodiment of the present invention is a use as described above, wherein said inhibitor of complex I is any of tritylthioalanine, carminomycin, and piperazinedione, rotenone, amytal, 1-methyl-4-phenylpyridinium, paraquat, methylene blue, or ferricyanide or a stereoisomer, tautomer, racemate, prodrug, metabolite thereof, or a pharmaceutically acceptable salt, base, ester or solvate thereof.

Another embodiment of the present invention is a use as described above, wherein said inhibitor of oxidative phosphorylation is an inhibitor of enzyme complex II (succinate-coenzyme Q reductase).

Another embodiment of the present invention is a use as described above, wherein said inhibitor of oxidative phosphorylation is an inhibitor of complex III (coenzyme Q cytochrome C reductase).

Another embodiment of the present invention is a use as described above, wherein said inhibitor of complex III is any of myxothiazol, antimycin A, ubisemiquinone, cytochrome C, 4,6-diaminotriazine derivatives, metothrexate, phenazine methosulfate and 2,6-Dichlorophenol-indophenol.

Another embodiment of the present invention is a use as described above, wherein said inhibitor of oxidative phosphorylation is an inhibitor of enzyme complex IV (cytochrome oxydase).

Another embodiment of the present invention is a use as described above, wherein said inhibitor of enzyme complex IV is any of cyanide, hydrogen sulfide, azide, formate, phosphine, carbon monoxide or electron acceptor ferricyanide or a stereoisomer, tautomer, racemate, prodrug, metabolite thereof, or a pharmaceutically acceptable salt, base, ester or solvate thereof.

Another embodiment of the present invention is a use as described above, wherein said inhibitor of oxidative phosphorylation is an inhibitor of enzyme complex V (F0-F1, ATP synthase).

Another embodiment of the present invention is a use as described above, wherein said inhibitor of enzyme complex V is any of 4′-demethyl-epipodophyllotoxin thenylidene glucoside, tritylthioalanine, carminomycin, piperazinedione, dinitrophenol, dinitrocresol, 2-hydroxy-3-alkyl-1,4-naphtoquinones, apoptolidin aglycone, oligomycin, ossamycin, cytovaricin, naphtoquinone derivatives, dichloroallyl-lawsone, lapachol, rhodamine, rhodamine 123, rhodamine 6G, carbonyl cyanide p-trifluoromethoxyphenylhydrazone, rothenone, safranine O, cyhexatin, dichlorodiphenyltrichloroethane, chlordecone, arsenate, pentachlorophenol, benzonitrile, thiadiazole herbicides, salicylate, cationic amphilic drugs, amiodarone, perhexiline, gramicidin, calcimycin, pentachlorobutadienyl-cysteine, trifluorocarbonylcyanide or phenylhydrazone or a stereoisomer, tautomer, racemate, prodrug, metabolite thereof, or a pharmaceutically acceptable salt, base, ester or solvate thereof.

Another embodiment of the present invention is a use as described above, wherein said inhibitor of oxidative phosphorylation is any of atractyloside, lysophospholipids, n-ethylmaleimide, mersanyl, or p-benzoquinone or a stereoisomer, tautomer, racemate, prodrug, metabolite thereof, or a pharmaceutically acceptable salt, base, ester or solvate thereof.

Another embodiment of the present invention is a use as described above, wherein said inhibitor of oxidative phosphorylation is any of rhodamine, rhodamine 6G, rhodamine 123, dinitrophenol, or rotenone or a stereoisomer, tautomer, racemate, prodrug, metabolite thereof, or a pharmaceutically acceptable salt, base, ester or solvate thereof.

Another embodiment of the present invention is a use as described above, wherein said oxidative phosphorylation inhibitor, when comprising a halogen atom, comprises a halogen radioisotope in place of a stable halogen atom.

Another embodiment of the present invention is a use as described above; wherein the oxidative phosphorylation inhibitor is present in an amount such that the concentration of inhibitor delivered to a subject is between 10 and 60 mg/kg.

One embodiment of the present invention is a use as described above, wherein said composition further comprises one or more agents to unlock or unblock the flow into the TCA cycle.

One embodiment of the present invention is a use as described above, wherein said agent to unlock the TCA cycle is serine or fructose 1-6 diP.

One embodiment of the present invention is a use as described above, wherein said composition further comprises one or more imaging agents

One embodiment of the present invention is a use as described above, wherein said imaging agent is any of MR visible polymer (such as poly(ortho)ester), metallic powder, tantalum powder, magnesium alloy, biocompatible metal powder, iridium powder, or micro-bubbles.

One embodiment of the present invention is a use as described above, further combined with radiotherapy.

One embodiment of the present invention is a use as described above, further combined with chemotherapy.

One embodiment of the present invention is a use as described above, wherein said composition further comprises one or more slow releasing polymers.

One embodiment of the present invention is a use as described above, wherein said slow release polymer is any of poly(glycolic) acid, poly(lactic acid) or in general glycolic- and lactic acid based polymers, copolymers, poly caprolactones and in general, poly hydroxyl alkanoate,s poly(hydroxy alcanoic acids), Poly (ethylene glycol), poly vinyl alcohol, poly (orthoesters), poly (anhydrides), poly (carbonates), poly amides, poly imides, poly imines, poly (imino carbonates), poly (ethylene imines), polydioxanes, poly oxyethylene (poly ethylene oxide), poly (phosphazenes), poly sulphones, lipids, poly acrylic acids, poly methylmethacrylate, poly acryl amides, poly acrylo nitriles (Poly cyano acrylates), poly HEMA, poly urethanes, poly olefins, poly styrene, poly terephthalates, poly ethylenes, poly propylenes, poly ether ketones, poly vinylchlorides, poly fluorides, silicones, poly silicates (bioactive glass), siloxanes (Poly dimethyl siloxanes), hydroxyapatites, lactide-capronolactone, poly aminoacids (natural and non natural), poly β-aminoesters, albumines, alginates, cellulose/cellulose acetates, chitin/chitosan, collagene, fibrine/fibrinogen, gelatine, lignine, proteine based polymers, Poly (lysine), poly (glutamate), poly (malonates), poly (hyaluronic acids), Poly nucleic acids, poly saccharides, poly (hydroxyalkanoates), poly isoprenoids, starch based polymers, copolymers thereof, linear, branched, hyperbranched, dendrimers, crosslinked, functionalised derivatives thereof.

One embodiment of the present invention is a use as described above, wherein said composition further comprises magnesium alloys. Said alloys may be used for imaging visibility or as a slow release agent.

One embodiment of the present invention is a use as described above, wherein at least one of said inhibitors is coupled to solubilising agent.

One embodiment of the present invention is a use as described above, solubilising agent is cholesterol or derivative thereof.

One embodiment of the present invention is a use as described above, wherein said cholesterol derivatives are any of cholesteryl-3-betahydroxybutyrate, cholesteryl-halogenated butyrate, cholesteryl-halogenated acetate, cholesteryl-halogenated aceto-acetate, cholesteryl-halogenated acetamide, cholesteryl-halogenated crotonate, cholesteryl-halogenated acetone, cholesteryl-halogenated citrate, or cholesteryl-halogenated oleate

One embodiment of the present invention is a use as described above, wherein solubilising agent is vitamin A or derivative thereof.

One embodiment of the present invention is a use as described above, wherein derivative of vitamin A is formula (IV) or (V) below wherein R is selected from the group consisting of betahydroxybutyrate, halogenated butyrate, halogenated acetate, halogenated aceto-acetate, halogenated acetamide, halogenated crotonate, halogenated acetone, halogenated citrate, and halogenated oleate.

One embodiment of the present invention is a use as described above, wherein at least one of said inhibitors is present in micro-capsule and/or nano-capsule.

One embodiment of the present invention is a use as described above wherein nano-capsule is any of copolymer poly(ethylene oxide) with poly(L-Lactic acid) or with poly(beta-benzyl-L-aspartate), copolymer with poly(lactide-co-glycolide)-[(propylene oxide)-poly(ethylene oxide)], polyphosphazene derivatives, poly(ethylene glycol) coated nanospheres, poly(isobutylcyanoacrylate) nanocapsules, poly(gamma-benzyl-L-glutamate)/(poly(ethylene oxide), chitosan-poly(ethylene oxide) nanoparticules, nanoparticules where said inhibitor is prepared using o-carboxymethylate chitosan as wall forming material, or solid lipid nanospheres (SLNs).

One embodiment of the present invention is a use as described above wherein micro-capsule is any of multiporous beads of chitosan, coated alginate microspheres, N-(aminoalkyl) chitosan microspheres, chitosan/calcium alginate beads, poly(adipic anhydride) microspheres, gellan-gum beads, poly(D,L-lactide-co-glycolide) microspheres, alginate-poly-L-lysine microcapsules, crosslinked chitosan microspheres, chitosan/gelatin microspheres, crosslinked chitosan network beads with spacer groups, 1,5-diozepan-2-one microspheres, D,L-dilactide microspheres, triglyceride lipospheres, polyelectrolyte complexes of sodium alginate chitosan, polypeptide microcapsules, or albumin microspheres.

One embodiment of the present invention is a use as described above, wherein said composition is administered by infusion into a mass of proliferating cells.

One embodiment of the present invention is a use as described above, wherein said composition is administered by high-pressure injection into a mass of proliferating cells.

One embodiment of the present invention is a use as described above, wherein said composition is administered by direct injection into a mass of proliferating cells.

Another embodiment of the present invention is a use as described above, wherein said composition is administered into a resection cavity or scar.

Another embodiment of the present invention is a use as described above, wherein said composition is part of a solid wall composition.

Another embodiment of the present invention is a use as described above, wherein said solid wall composition is a capsule of suitable size and shape for administration using a needle, said capsule filled with composition.

Another embodiment of the present invention is a use as described above, wherein a wall of said capsule comprises gelatin.

Another embodiment of the present invention is a use as described above, wherein said solid wall composition is a solid state bioabsorbable structure of suitable size and shape for administration using a needle, said structure impregnated with composition.

Another embodiment of the present invention is a use as described above, wherein said solid state bioabsorbable structure is seed-shaped, rod-shaped, or tube-shaped.

One embodiment of the present invention is a kit comprising a composition comprising one or more inhibitors of the TCA cycle and/or one or more inhibitors of oxidative phosphorylation.

One embodiment of the present invention is a kit as described above wherein said composition is a composition as mentioned above.

One embodiment of the present invention is a kit as described above, further comprising a syringe.

One embodiment of the present invention is a hydrogel comprising a) composition as defined above, and b) an activated polyethyleneglycol (PEG) combined with any of alkaline hydrolyzed soya solutions, animal or vegetal proteins, bovine serum albumin, soya globulin, casein, pea albumin, starch albumine, or ovalbumin.

One embodiment of the present invention is a hydrogel as described above wherein a TCA inhibitor of the composition is present at a concentration of less than or equal to 0.1 mg per square cm of hydrogel and/or an oxidative phosphorylation inhibitor of the composition is present at a concentration of less than or equal to 1 mg per square cm of hydrogel.

One embodiment of the present invention is a use of a hydrogel as described above for treatment of superficial cell proliferation, such as basal carcinoma or a squamous cell carcinoma by application of the hydrogel to the surface of said proliferations.

### DETAILED DESCRIPTION OF THE INVENTION

Unless defined otherwise, all technical and scientific terms used herein have the same meaning as is commonly understood by one of skill in the art. All publications referenced herein are incorporated by reference thereto. All United States patents and patent applications referenced herein are incorporated by reference herein in their entirety including the drawings.

The articles “a” and “an” are used herein to refer to one or to more than one, i.e. to at least one of the grammatical object of the article. By way of example, “a TCA cycle inhibitor” means one inhibitor or more than one inhibitor.

Throughout this application, the term “about” is used to indicate that a value includes the standard deviation of error for the device or method being employed to determine the value.

The recitation of numerical ranges by endpoints includes all integer numbers and, where appropriate, fractions subsumed within that range (e.g. 1 to 5 can include 1, 2, 3, 4 when referring to, for example, a number of TCA cycle inhibitors, and can also include 1.5, 2, 2.75 and 3.80, when referring to, for example, dose concentrations).

The present invention relates to the use of substances that block the citric acid cycle (TCA cycle) and optionally oxidative phosphorylation, for the treatment of the proliferation of cells.

The present invention also relates to a method for the treatment of proliferating cells present in a subject, comprising administering into the proliferating cell mass a composition comprising at least one type of the TCA cycle and/or oxidative phosphorylation.

The present invention also relates to a method for sensitising proliferating cells present in a cavity of a subject to treatment by radiotherapy and/or chemotherapy, comprising administering into the proliferating cell mass a composition comprising at least one type of the TCA cycle and/or oxidative phosphorylation prior to said radiotherapy and/or chemotherapy.

Proliferative or proliferating cells, whether benign or malignant, are cells such as cancer cells, vascular restenosis cells, hypertrophic scar cells, cheloid scar cells, inflammatory cells, benign tumor cells or any rapidly proliferating cell. Such cells are found in, for example, tumours, hyperproliferative scars, cheloid scars, myoma or fibroma benign tumors and restenotic processes or tumors at the level of a duct. Generally such cells rapidly proliferate (hyperproliferative). A collection of such cells form a cell mass.

Magnetic resonance spectroscopy studies performed by the inventors have shown the TCA cycle is highly active in a majority of hyperproliferative cells, compared with adjacent cells not undergoing hyperprofileration. This property enables one or more TCA cycle inhibitors to be employed proximal to the site of cell proliferation, and to be rapidly taken up by said proliferating cells in doses higher than by non-proliferating cells. Consequently high doses of inhibitor or extremely potent inhibitors may be applied locally, resulting in cell death of hyperproliferative cells, and reduced or no cell death of non-proliferating cells. The differential in TCA metabolism can allow for a reduction in the amount of active substance necessary compared with conventional chemotherapy.

Furthermore, the inventors have realised that inhibition of one or more enzymes in the TCA cycle alone can lead to cell death. Surprisingly, other pathways do not compensate effectively for the inhibition of one or more enzymes. The inventors have also found that even further cell death is achieved when oxidative phosphorylation is additionally inhibited.

One embodiment of the present invention is a use of a composition comprising one or more inhibitors of the TCA cycle and optionally one or more inhibitors of oxidative phosphorylation for treating hyperproliferative cells administered to or in the region of the proliferation, preferably into the proliferating cell mass.

One embodiment of the present invention is a use of a composition comprising one or more inhibitors of the TCA cycle and optionally one or more inhibitors of oxidative phosphorylation for the preparation of a medicament for treating hyperproliferative cells, wherein the composition is administered to or in the region of the proliferation, preferably into the proliferating cell mass.

One embodiment of the present invention is a method for the treatment of cellular proliferation comprising administering to a patient a composition comprising one or more inhibitors of the TCA cycle and optionally one or more inhibitors of oxidative phosphorylation for treating hyperproliferative cells, said administration to or in the region of the proliferation, preferably into the proliferating cell mass.

The present invention also relates to the use of substances that inhibit oxidative phosphorylation, for the treatment of the proliferation of cells.

One or more oxidative phosphorylation inhibitors may be employed proximal to the site of cell proliferation, to be rapidly taken up by proliferating cells in doses higher than by non-proliferating cells. Consequently high doses of inhibitor or extremely potent inhibitors may be applied locally, resulting in cell death of hyperproliferative cells, and reduced or no cell death of non-proliferating cells. The differential in oxidative phosphorylation metabolism can allow for a reduction in the amount of active substance necessary compared with conventional chemotherapy.

One embodiment of the present invention is a use of a composition comprising one or more inhibitors of oxidative phosphorylation for treating hyperproliferative cells administered to or in the region of the proliferation, preferably into the proliferating cell mass.

One embodiment of the present invention is a use of a composition comprising one or more inhibitors of oxidative phosphorylation for the preparation of a medicament for treating hyperproliferative cells, wherein the composition is administered to or in the region of the proliferation, preferably into the proliferating cell mass.

One embodiment of the present invention is a method for the treatment of cellular proliferation comprising administering to a patient a composition comprising one or more inhibitors of oxidative phosphorylation for treating hyperproliferative cells, said administration to or in the region of the proliferation, preferably into the proliferating cell mass.

The present invention is useful for treating cellular proliferation in any animal including humans, livestock, domestic animals, wild animals, or any animal in need of treatment. Examples of an animal is human, horse, cat, dog, mice, rat, gerbil, bovine species, pig, fowl, camelidae species, goat, sheep, rabbit, hare, bird, elephant, monkey, chimpanzee etc. An animal may be a mammal.

The composition can be a pharmaceutical composition. Where a particular use of a composition of the present invention is described, said use may be understood as a method. In the preferred mode of the invention, an inhibitor of oxidative phosphorylation is rhodamine (i.e. rhodamine, rhodamine 6G, rhodamine 123). In another preferred mode of the invention an inhibitor of the TCA cycle is fluoroacetate. The present invention may be applied using any inhibitors of these pathways as indicated below. The composition may be administered to or in the region of the proliferation. Thus, composition is therefore, administered locally to the site of proliferation, and not systemically. Preferably it is administered into the proliferating cell mass.

Bone Metastasis

The composition may be used to treat a naive (not previously treated) benign or malignant cell proliferation by direct intratumoral administration, infusion or injection. The composition could also be used to treat the metastatis originating from a malignant tumour. Metastasis are masses originating from malignant cells which have travelled in the body through blood or lymphatic vessels. They may develop in almost any place in the body: brain, lung, liver, bone, etc. All these metastatic areas may be treated using the same composition. For instance, the standard therapy for a bone metastatis is local radiotherapy. A more recent treatment is cementoplasty, where a polymeric cement (methylmetacrylate for instance) is injected directly inside the tumor area in order to avoid its fracture, in place where such fracture may have heavy consequences (vertebra). In both cases, before local radiotherapy or cementoplasty is performed, one could inject inside the bone the active composition.

Another embodiment of the present invention is a composition as described herein further comprising pyrophosphate. Said composition may be used to treat a bone tumour first, by preventing tumour cell proliferation, and in a second step, to stimulate bone reconstruction. The composition may be injected into a bone metastasis or cavity left after removal of a bone cancer. According to the present pyrophosphate may be any suitable salt of pyrophosphate, including, but not limited to sodium pyrophosphate, potassium pyrophosphate, calcium pyrophosphate The pyrophosphates may be mixed to the polymer containing the inhibitors. Once the polymer has been degraded and all inhibitors have been absorbed, the presence of pyrophosphates may stimulate new bone formation.

TCA Cycle Inhibitors

A TCA cycle inhibitor of the invention is any inhibitor of one or more enzymes of the TCA cycle. The TCA cycle enzymes are known in the art and include pyruvate dehydrogenase, citrate synthase, aconitase, isocitrate lyase, alpha-ketoglutarate dehydrogenase complex, succinyl CoA synthetase, succinate dehydrogenase, fumarase, malate synthase, glutaminase, and pyruvate dehydrogenase complex.

Inhibitors of pyruvate dehydrogenase are any known in the art and may include, but are not limited to any of arsenite, dichlorovinyl-cysteine, p-benzoquinone, thiaminase.

Inhibitors of citrate synthetase are any known in the art and may include, but are not limited to any of the following:

Fluoroacetate (an its derivative fluoroacetyl-CoA), any halogenated acetyl-CoA, fluoroacetamide, fluorocrotonate, halogenated ketone bodies (for instance, chloroacetoacetate, fluoroacetoacetate, fluorohydroxybutyrate, chlorohydroxybutyrate, bromohydroxybutyrate), halogenated acetone, halogenated acetic acid (for example chloracetic acid), halogenated oleate (an analogue of ketone bodies) and any known in the art.

Inhibitors of aconitase are any known in the art and may include, but are not limited to any of the following:

Fluorocitrate, fluorocitrate 2R, 3R, and any other halogenated citrate (bromocitrate, chlorocitrate).

Inhibitors of isocitrate dehydrogenase are any known in the art and may include, but are not limited to any of the following:

DCVC (dichlorovinyl-cysteine)

Inhibitors of succinate dehydrogenase are any known in the art and may include, but are not limited to malonate, DCVC, Pentachlorobutadienyl-cysteine (or PCBD-cys), 2-bromohydroquinone, 3-nitropropionic acid, cis-crotonalide fungicides.

Inhibitors of succinyl CoA synthetase, alpha ketoglutarate dehydrogenase complex, fumarate hydratase (fumarase), malate dehydrogenase are any known in the art.

Inhibitors of glutaminase are any known in the art and may include, but are not limited to 6-diazo-5-oxo-L-norleucine (DON).

Inhibitors of glutamate dehydrogenase are any known in the art.

Other inhibitors of the TCA cycle include glu-hydroxyoxamate, p-chloromercuriphenylsulphonic acid (impermeant thiol agent), L-glutamate gamma-hydroxamate, p-chloromercuriphenylsulphonic acid, acivicin (alpha-amino-3-chloro-4,5 dihydro-5-isoxazoleacetic acid), halogenated glutamine and glutamate.

According to another embodiment of the invention, a TCA cycle inhibitor is any of arsenite, hypoglycin A, methylenecyclopropylacetic acid, alloxan, PNU, p-benzoquinone, fluoroacetate, halogenated acetates (iodo-, bromo-, chloro-acetate), halogenated acetyl-CoA (fluoroacetyl-CoA, bromoacetyl-CoA, chloroacetyl-CoA, iodoacetyl-CoA), halogenated crotonate (fluoro-, iodo-, bromo-, chloro-crotonate), halogenated ketone bodies, (chloro-, fluoro-, bromo-, iodoaceto-acetate, fluoro-, chloro-, bromo-, iodo-butyrate, fluoro-, chloro-, bromo-, iodo-acetone), halogenated oleate (iodo, bromo, chloro, fluoro-oleate), halogenated citrate, halogenated citrate 2R, 3R isomer (fluoro-, bromo-, chloro-, iodo-citrate), dichlorovinyl-cysteine, halogenated aminoacids, malonate, pentachlorobutadienyl-cysteine, 2-bromohydroquinone, 3-nitropropionic acid, cis-crotonalide fungicides, glu-hydroxyoxamate, p-chloromercuriphenylsulphonic acid, L-glutamate gamma-hydroxamate, p-chloromercuriphenylsulphonic acid, acivicin (alpha-amino-3-chloro-4,5-dihydro-5-isoxazoleacetic acid, halogenated glutamine (fluoro, iodo, chloro, bromo-glutamine), or halogenated glutamate (fluoro, iodo, chloro, bromo-glutamate).

Where more than one inhibitor of the TCA is present in a composition, preferably, one inhibitor is directed towards the upper half of the TCA cycle, which is characterised by providing no redox products such as NADH, HANPH, or FADH₂ (e.g. enzymes pyruvate dehydrogenase, citrate synthase, aconitase) and another inhibitor is directed towards the lower half of the TCA cycle, which is characterised by providing redox products such as NADH, HANPH, or FADH₂ (e.g. enzymes isocitrate lyase, alpha-ketoglutarate dehydrogenase complex, succinyl CoA synthetase, succinate dehydrogenase, malate synthase, glutaminase). Examples of a combination of inhibitor includes fluoroactetate and malonate.

Fluorocitrate and Derivatives

According to a preferred embodiment of the invention, a TCA cycle inhibitor of the invention has a formula (I): 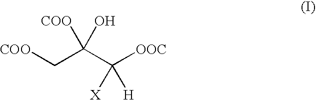
where X may be halide, a sulfonate, a carboxylate, an alkoxide, an amine oxide or a OH. The halide may be selected from the group consisting of: fluoride, bromide, chloride, and iodide. The sulfonate may be selected from the group consisting of: triflate, mesylate and tosylate. The carboxylate may be selected from the group consisting of: methoxylate and ethyloxylate. The alkoxide may be selected from the group consisting of: methoxide and ethoxide. The amine oxide is dimethylamine oxide. According to one aspect of the invention, the stereochemistry is 2R, 3R.
Fluoroacetate and Derivatives

TCA cycle inhibitors also includes substances which are converted into inhibitors of the TCA cycle such as, for example fluoroacetate and derivatives. According to a preferred embodiment of the invention, a TCA cycle inhibitor of the invention has a formula (II): 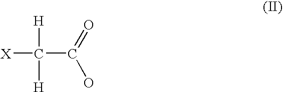
where X may be halide, a sulfonate, a carboxylate, an alkoxide, or an amine oxide, a OH. The halide may be selected from the group consisting of: fluoride, bromide, chloride, and iodide. The sulfonate may be selected from the group consisting of: triflate, mesylate and tosylate. The carboxylate may be selected from the group consisting of: methoxylate and ethyloxylate. The alkoxide may be selected from the group consisting of: methoxide and ethoxide. The amine oxide may be dimethylamine oxide.

Preferably, an inhibitor of the TCA cycle is any of fluoroacetate, fluorocitrate, arsenite, acetoacetate, and betahydroxy butyrate.

The requirement for both energy and building blocks such as lipids, amino acids, nucleic acids by rapidly proliferating cells means the TCA cycle is highly active. The inventors have found that the cells become susceptible to uptake of substrates of the TCA cycle such as acetate, much more so than non-proliferating cells. Therefore, treatment using the invention benefits from rapid and selective inhibitor uptake, and cell death owing to the inhibition of the central anabolic pathway. More efficient cell death may be achieved in combination with other energy-producing pathways such as glycolysis and oxidative phosphorylation. Simultaneous inhibition of the PPP also shuts down an important anabolic pathway

Halogenated Inhibitors Which Are Radioisotopes

According to an aspect of the present invention, at least one halogen atom where present in an inhibitor of the TCA cycle is substituted for the corresponding halogen atom radioisotope, to form a radio-isotope-halogen TCA cycle inhibitor (RIH-TCA cycle inhibitor). The radioisotopes of halides may be for example ¹⁸F, ⁷⁹Br, ⁸¹Br, ³⁶Cl, ¹²⁹I, ¹²⁵I, ¹³¹I, which all emit ionising radiation. For example, the stable fluorine atom of fluoroacetate may be substituted for ¹⁸F to form ¹⁸F-fluoroacetate. Similarly ¹²⁵I-iodoacetate may be employed as a TCA cycle inhibitor. The use of an RIH-TCA cycle inhibitor allows effective brachytherapy, simultaneously with pathway inhibition as described above. Furthermore, where used in combination therapy the RIH-TCA cycle inhibitor can be administered sequentially after an oxidative phosphorylation inhibitor; the proliferating cells, therefore, are affected both in terms of energy production by the inhibitors and by the ionising radiation of the RIH-TCA cycle inhibitor. The dose of the RIH-TCA cycle inhibitor can be adjusted so that the cytotoxic effect is due to the radiation rather than pathway inhibition or vice versa.

Simultaneous Inhibition of Other Pathways

According to an aspect of the invention a TCA cycle inhibitor is capable of inhibiting at least 3 cellular mechanisms of proliferating cells simultaneously. This may be achieved by blocking, for example, aconitase from the TCA cycle. The inventors have realised that the use of an aconitase inhibitor such as, for example, fluorocitrate (or fluoroacetate which is later converted into fluorocitrate) can inhibit other important pathways such as fatty acid synthesis at the level of ATP-citrate lyase and calcium intracellular signalling through derivatives accumulation.

Oxidative Phosphorylation Inhibitors

An oxidative phosphorylation inhibitor of the invention is any inhibitor of one or more enzymes of oxidative phosphorylation. The oxidative phosphorylation enzymes are known in the art and include enzyme complex I (NADH coenzyme Q reductase), II (succinate-coenzyme Q reductase), III (coenzyme Q cytochrome C reductase), IV (cytochrome oxydase), and V (F0-F1, ATP synthase).

Inhibitors of enzyme complex I are any known in the art and may include, but are not limited to any of the following: tritylthioalanine, carminomycin, and piperazinedione, rotenone, amytal, 1-methyl4-phenylpyridinium (MPP+), paraquat, methylene blue, Ferricyanide (the later 2 are electron acceptors).

Inhibitors of enzyme complex II are any known in the art.

Inhibitors of coenzyme Q are any known in the art.

Inhibitors of enzyme complex III are any known in the art and may include, but are not limited to myxothiazol, antimycin A, ubisemiquinone, cytochrome C, 4,6-diaminotriazine derivatives, metothrexate or electron acceptors such as phenazine methosulfate and 2,6-Dichlorophenol-indophenol.

Inhibitors of enzyme complex IV are any known in the art and may include, but are not limited to cyanide, hydrogen sulfide, azide, formate, phosphine, carbon monoxide and electon acceptor ferricyanide.

Inhibitors of enzyme complex V are any known in the art and may include, but are not limited to VM-26 (4′-demethyl-epipodophyllotoxin thenylidene glucoside), tritylthioalanine, carminomycin, piperazinedione, dinitrophenol, dinitrocresol, 2-hydroxy-3-alkyl-1,4-naphtoquinones, apoptolidin aglycone, oligomycin, ossamycin, cytovaricin, naphtoquinone derivatives (e.g. dichloroallyl-lawsone and lapachol), rhodamine, rhodamine 123, rhodamine 6G, carbonyl cyanide p-trifluoromethoxyphenylhydrazone, rothenone, safranine O, cyhexatin, DDT, chlordecone, arsenate, pentachlorophenol, benzonitrile, thiadiazole herbicides, salicylate, cationic amphilic drugs (amiodarone, perhexiline), gramicidin, calcimycin, pentachlorobutadienyl-cysteine (PCBD-cys), trifluorocarbonylcyanide phenylhydrazone (FCCP).

Other inhibitors of oxidative phorphorylation may include atractyloside, DDT, free fatty acids, lysophospholipids, n-ethylmaleimide, mersanyl, p-benzoquinone.

According to an embodiment of the invention, a oxidative phosphorylation inhibitor is any of rotenone, amytal, 1-methyl-4-phenylpyridinium, paraquat, myxothiazol, antimycin A, ubisemiquinone, cytochrome C, 4,6-diaminotriazine derivatives, cyanide, hydrogen sulfide, azide, formate, phosphine, carbon monoxide, 4′-demethyl-epipodophyllotoxin thenylidene glucoside, tritylthioalanine, carminomycin, piperazinedione, dinitrophenol, dinitrocresol, 2-hydroxy-3-alkyl-1,4-naphtoquinones, apoptolidin aglycone, oligomycin, ossamycin, clofazimine cytovaricin, naphtoquinone derivatives, dichloroallyl-lawsone, lapachol, rhodamine, rhodamine 123, rhodamine 6G, carbonyl cyanide p-trifluoromethoxyphenylhydrazone, cyhexatin, dichlorodiphenyltrichloroethane (DDT), chlordecone, arsenate, pentachlorophenol, benzonitrile, thiadiazole herbicides, salicylate, cationic amphilic drugs, amiodarone, perhexiline, gramicidin, calcimycin, pentachlorobutadienyl-cysteine, trifluorocarbonylcyanide phenylhydrazone, atractyloside, lysophospholipids, n-ethylmaleimide, mersanyl, or p-benzoquinone.

Preferably, an inhibitor of oxidative phosphorylation is any of rhodamine (i.e. rhodamine, rhodamine 6G, rhodamine 123), dinitrophenol, or rotenone.

Where rhodamine, rhodamine 123, or rhodamine 6G are present in the composition, they may be used as inhibitors of oxidative phosphorylation, and not as a dye for homogeneous coating control, or for photodynamic therapy, for instance. Therefore, where rhodamine compounds are used, the treatment according to the invention is not in combination with rhodamine-based imaging or light-based treatment.

Inhibition of oxidative phosphorylation surprisingly leads to an inhibition of proliferation; proliferating cells do not obtain energy via other pathways and recover. The inventors have also found that inhibition of both oxidative phosphorylation and TCA cycle is further effective against proliferating cells. While in vitro data shows rhodamine inhibition of cancer cells growth, in vivo, rhodamine given by intravenous route seems less efficaceous to treat tumours such as prostate cancer. The Huntington Medical Research Institutes are addressing this issue in metastatic prostate cancer (Lawrence W. Jones, M. D. Huntington Memorial Hospital). Recently this institute published the results of a clinical trial where 33 patients with metastatic prostate cancer were treated with Rhodamine 123 intra-venous infusions. A slight PSA reduction was seen in some cases, but no significant tumour response was noted (Jones L W, Narayan K S, Shapiro C E, Sweatman T W. Rhodamine-123: Therapy for hormone refractory prostate cancer. A phase I clinical trial. Journal of Chemotherapy. 17: 435-440, 2005).

Because inhibitors of oxidative phorphorylation are extremely toxic to a subject in the doses needed for efficacy via the systemic route, such inhibitors have been largely overlooked for effective treatment of conditions such as cancer. For example, it is known that the LD10 (the dose which kills 10% of animals) for rhodamine 123 in rodents is 20 mg/kg. It is also known that the maximal tolerated dose of Rhodamine 123 delivered intra-venously in man is 95 mg/m². For a 70 kg patient, the maximum dose of systemically delivered rhodamine is about 150 mg. Operating at the maximum, non-lethal dose, clinical trials have shown no useful effect on prostate tumours. There was an accumulation of rhodamine in the prostate of the patients, but it was not sufficient to provoke a significant response.

By locally delivering oxidative phosphorylation inhibitors into proliferating cells in a slow release formulation, the delivery period is prolonged i.e. the inhibitors are not cleared by the liver, and the dose received by the tumour is effectively higher than using systemic delivery. Furthemore, the lethal dose can be greatly exceeded. For instance delivering 40 or 50 mg of rhodamine directly in the tumour leads to tumour death, and not just to a slow down in tumour growth. Such a dose, delivered systemically, would require 100 g of rhodamine, more than 1000 times the lethal dose. Thus, a composition comprising an oxidative phorphosrylation inhibitor in a slow release formulation increases the effective dose to the tumour and permits greater than lethal dosing.

Furthermore, the composition comprising an oxidative phosphorylation inhibitor may be used in combination with radiotherapy, where a lower dose of inhibitor may be used.

Halogenated Inhibitors Which Are Radioisotopes

According to an aspect of the present invention, at least one halogen atom where present in an inhibitor of oxidative phosphorylation is substituted for the corresponding halogen atom radioisotope, to form a radio-isotope-halogen oxidative phosphorylation cycle inhibitor (RIH-oxidative phosphorylation inhibitor). The radioisotopes of halides may be for example ¹⁸F, ⁷⁹Br, ⁸¹Br, ³⁶Cl, ¹²⁵I, ¹²⁹I, ¹³¹I, which all emit ionising radiation. For example, a stable fluorine atom of pentachlorophenol may be substituted for ¹⁸F to form ¹⁸F-pentachlorophenol. The use of an RIH-oxidative phosphorylation inhibitor allows effective brachytherapy, simultaneously with pathway inhibition as described above. Furthermore, where used in combination therapy, the RIH-oxidative phosphorylation inhibitor can be administered sequentially after a TCA cycle inhibitor; the proliferating cells, therefore, are affected both in terms of energy production by the inhibitors and by the ionising radiation of the RIH-oxidative phosphorylation inhibitor. The dose of the RIH-oxidative phosphorylation inhibitor can be adjusted so that the cytotoxic effect is due to the ionising radiation rather than pathway inhibition, or vice-versa.

Combination of Pathway Inhibitors

The present invention not only encompasses a composition comprising either an inhibitor of TCA or an inhibitor of oxidative phosphorylation, but also a combination of both TCA inhibitor and of oxidative phosphorylation inhibitor.

Experimental data led the inventors combine both TCA and oxidative phosphorylation inhibition for more effective treatment of cell proliferation. Indeed, the 3 main cycles involved in ATP production, namely glycolysis, TCA and oxphos are interconnected by numerous pathways (shuttles), which allow circumvention of a pathway when it is inactivated (Biochemistry, Third edition, Mathews C K et al, Addison Wesley Longman, Inc, 2000).

Literature data as well as inventor's own observations using Positron Emission Tomography (PET) show that some tumours will use glucose more readily, others will rely more on acetate, while others will rely mainly on glutamine. This means there is an interest to combine ATP inhibitors to be to disable one to several mechanisms of cell proliferation. The inventors also noted that the phenotype of energy consumption of a tumour compared with its metastasis may change, i.e. phenotypic mutations may occur inside the same cell family when the tumour cell has migrated. For instance, the “mother” tumour may intake ¹¹C-acetate (PET tracer) while some of its metastases will not. PET CT examinations using these different “contrast agents” (18-FDG, 11C-acetetate, etc.) may be performed in all patients in order to detect individual affinity of primitive tumours and metastases for the best absorbed element. The inventors have also noted that the TCA cycle is activated in various proportions in tumours. Some tumours will augment their TCA cycle by 20%, while in other the TCA cycle is augmented by 200%.

A known combination blocking cell proliferation associates a glycolysis inhibitor (2-deoxyglucose or 2-DG) with an oxidative phosphorylation inhibitor (rhodamine 123) (Bernal S D, Adenocarcinoma activity of Rhodamine 123, a mitochondrial specific dye, Science 222, 169-171, 1983). In this combination rhodamine 123 uncouples oxidative phosphorylation, (i.e. ATP synthesis by the respiratory chain of mitochondria is blocked), so the production of ATP relies on glycolysis. Blocking glycolysis stops cell proliferation.

In tumour cells, glycolysis is stimulated—some metabolites of glycolysis are intermediates in the manufacture of nucleotides (PPP pathway), some are used to manufacture ATP. At the end of the glycolysis chain, some remaining metabolites end up in lactate production. TCA is increased also in tumour cells, sometimes moderately, by 20-30%, sometimes much more, by several hundred %, depending on proliferating cell “energy consumption phenotype”.

In view of the inventor's own experimental observation, an alternative control of the energy pathway is described here. It is important to understand that blocking oxidative phosphorylation (for instance by uncoupling, using rhodamine 123, dinitrophenol, etc.) does not mean the cell has become anaerobic or hypoxic. The O₂ may still be used by the tumour cell as normally, but the production of ATP by mitochondria is impossible because of an uncoupling, when complex V is inhibited. As TCA is oxygen dependent, it continues to function normally or is slightly diminished. Shuttles around TCA are activated and some metabolites may go for instance into fatty acid synthesis, glutaminolysis may be activated, etc.

The choice of blocking TCA and oxidative phosphorylation in combination leads to inhibition of several pathways simultaneously, creating combined and additional toxicity. For instance, the inhibition of TCA by fluoroacetate inhibits the TCA, fatty acid synthesis; it also has some negative feedback control of glycolysis by fluorocitrate accumulation, Ca²⁺ accumulation, etc. It is a very strong inhibition because resulting fluorocitrate cannot be processed by aconitase. In addition, the blockage of the mitochondrial activity reduces the use of metabolites and decreases the production of ATP, creating additional toxicity in another energy pathway of the cell. The combination of both inhibitors allows blocking as many pathways as possible and to avoid various biochemical shuttles overcoming the inhibition.

Some particular combinations of TCA and oxidative phosphorylation inhibitors injected directly inside a proliferating structure will present additional advantages. The inventors have observed the active accumulation of ¹¹C-acetate in several types of benign proliferating cells as well as in neoplasms (PET CT examinations). It is also known that rhodamine 123 accumulates for prolonged periods in dividing cells, as a result of high membrane potential across the mitochondrial membrane. Some combinations benefit from the fact that inhibitors do not just passively diffuse, but are actively absorbed by proliferating cells. Substances such as monofluoroacetate and rhodamine 123, when injected locally in a slow release presentation, are present at a high concentration in the target, slowly elute the toxic compounds, which are accumulating actively inside proliferating cells mainly. The accumulation of local injection, slow release, active uptake by proliferating cells, combined with high drug toxicity resulting from complementary effects, will allow to decrease the doses of each drug locally and for the whole body, possibly reducing side effects.

The concept was validated by the inventors in patients; inventors have treated some gynecologic tumours by injecting 2 mg of fluoroacetate and 20 mg of rhodamine in a slow release formulation in tumours weighting from 10 to 30 gr, which has led to a total tumor necrosis.

The combination of ATP inhibitors is very important for the inventors. The inventor's own clinical experience (MR spectroscopy and PET-CT examinations) as well as data from the literature confirm the great variety of substrates taken up by tumours. A review of the literature data shows for instance that the affinity for 18-FDG intake varies from 3 to 100%, depending on evaluated tumours and affected organs (e.g. http://www.petscaninfo.com/zportal/portals/phys/clinical/jnmpetlit/index_-html/JNM_OncoApps/JNM_Table8/article_elements_view). The inventors have also observed that a beneficial therapeutic approach is to treat several ATP synthesis pathways simultaneously.

Simultaneous, Separate, Sequential

According to one aspect of the invention, an oxidative phosphorylation inhibitor may be administered simultaneous, separate or sequentially in respect of a TCA cycle inhibitor of the invention.

Another aspect of the invention is a composition comprising at least one TCA cycle inhibitor as disclosed herein and at least one oxidative phosphorylation inhibitor, for simultaneous, separate or sequential administration to a subject.

One aspect of the invention is a method for treating cellular proliferation comprising administering to an individual an effective amount of at least one TCA cycle inhibitor of the invention and at least one oxidative phosphorylation inhibitor, simultaneously, separately or sequentially.

By simultaneous administration means the TCA cycle inhibitor and oxidative phosphorylation inhibitor are administered to a subject at the same time. For example, as a mixture or a composition comprising said components. An example is as a solution comprising the components of interest.

By separate administration means the TCA cycle inhibitor and oxidative phosphorylation inhibitor are administered to a subject at the same time or substantially the same time. The components may be present in a kit as separate, unmixed preparations. For example, the TCA cycle inhibitor and oxidative phosphorylation may be present in the kit as individual vials. The inhibitors may be administered to the subject by separate injections at the same time, or injection directly following the other.

For example, in a prostate cancer, 12 punctures may be performed in the peripheral area of the prostate. One out of each second puncture is made with a composition comprising a slow release formulation of fluoroacetate, and the other puncture is performed with a composition comprising a slow release formulation of dinitrophenol, rhodamine 123, rhodamine 6G, or malonate. Injection sessions may be repeated several times, depending on PSA levels.

In another example, a slow release formulation of an oxidative phosphorylation inhibitor may be injected inside a proliferative process such as a tumor, and a preparation of TCA inhibitors may be delivered through an infusion at the same time or a few days later.

Both therapeutic substances may also be mixed in a same slow release formulation. For instance, for prostate cancer treatment, it is also possible to administer one formulation first, and the other formulation later on, if PSA levels do not come down completely after the first therapy.

By sequential administration means the TCA cycle inhibitor and oxidative phosphorylation inhibitor are administered to a subject sequentially. The TCA cycle inhibitor and oxidative phosphorylation inhibitor may be present in a kit as separate, unmixed preparations. There is a time interval between doses. For example, one component might be administered up to 336, 312, 288, 264, 240, 216, 192, 168, 144, 120, 96, 72, 48, 24, 20, 16, 12, 8, 4, 2, 1, or 0.5 hours after the other component.

Taking the above mentioned example again, the 12 punctures around a prostrate cancer may first be made with a composition comprising a slow release formulation of fluoroacetate. Later on other punctures are performed with a composition comprising a slow release formulation of dinitrophenol, rhodamine 123, rhodamine 6G, or malonate, if PSA levels do not come down completely after the first therapy.

In sequential administration, one component may be administered once, or any number of times and in various doses before and/or after administration of another component. Sequential administration may be combined with simultaneous or sequential administration.

Derivatives

Stereoisomer, tautomers, racemates, prodrugs, metabolites, pharmaceutically acceptable salts, bases, esters, structurally related compounds or solvates of TCA cycle or oxidative phosphorylation inhibitors are within the scope of the invention.

The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such a sarginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

The term “stereoisomer”, as used herein, defines all possible compounds made up of the same atoms bonded by the same sequence of bonds but having different three-dimensional structures which are not interchangeable, which the compounds of the present invention may possess. Unless otherwise mentioned or indicated, the chemical designation of a compound herein encompasses the mixture of all possible stereochemically isomeric forms, which said compound may possess. Said mixture may contain all diastereomers and/or enantiomers of the basic molecular structure of said compound. All stereochemically isomeric forms of the compounds of the invention either in pure form or in admixture with each other are intended to fall within the scope of the present invention.

The compounds according to the invention may also exist in their tautomeric forms. Such forms, although not explicitly indicated in the compounds described herein, are intended to be included within the scope of the present invention.

For therapeutic use, the salts of the compounds according to the invention are those wherein the counter-ion is pharmaceutically or physiologically acceptable.

As used herein and unless otherwise stated, the term “solvate” includes any combination which may be formed by a compound of this invention with a suitable inorganic solvent (e.g. hydrates) or organic solvent, such as but not limited to alcohols, ketones, esters and the like.

The term “pro-drug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs of the compounds of the invention can be prepared by modifying functional groups present in said component in such a way that the modifications are cleaved, either in routine manipulation or in vivo, to the parent component. Typical examples of pro-drugs are described for instance in WO 99/33795, WO 99/33815, WO 99/33793 and WO 99/33792 all incorporated herein by reference. Pro-drugs are characterized by increased bio-availability and are readily metabolized into the active inhibitors in vivo. Specific examples of prodrugs comprising cholesterol or vitamin A are described below.

Administration

Administering to the region of the proliferation avoids or minimises peripheral toxicity, and permits delivery of efficacious doses. It also avoids toxicity associated with oral or systemic delivery. Methods of local delivery are known in the art.

According to one embodiment of the invention, the composition is administered into the proliferating tissue. The composition can enter the tissue, for example, by puncturing the surface of the tumour and injecting composition therein. Such administration is achieved by positive pressure application, for example, by high or low pressure injection, etc as described below. Alternatively, it may be achieved by intra-arterial infusion as also described below. Alternatively, the composition may enter the tissue after opening, for example, by resection surgery to remove the profilerating cell mass, as elaborated below.

According to one embodiment of the invention, a composition is administered under positive pressure to a proliferating tissue. Devices suitable for pressured delivery are known in the art, for example from US 2001/0034503 which is incorporated herein by reference. The latter document describes an apparatus for injecting a composition into a tissue under high pressure through a thin catheter (200 microns outer diameter for instance) with very small lateral holes (50 microns) located at the level of the tissue. One may leave the catheter inside the proliferating tissues for several days, and inject determined amounts of substances directly inside the proliferating structure at regular intervals allowing effective penetration of tissue. Where the proliferating cells form a large tissue mass, for example, a pressure between 2 to 5000 Atm. may used to apply the composition, leading to effective distribution of several centimetres radius within the tissue mass. Such injections are rapidly administered in a single shot. As the injected substances may be very toxic (fluorocitrate, arsenate, cyanide), it is important to be able to modulate the injected amounts very precisely as it is the case with this device. According to an aspect of the invention, a quantity of composition between 1 microlitre and 100 ml is injected at a pressure between 100 and 2500 Atm. The treatment may be repeated at defined intervals or as necessary.

According to another embodiment of the invention, a composition is delivered locally, preferably into the proliferating cell mass, under pressure by means of a continuous pump. A pump may be used to inject composition over a period of time, from several hours to several days as necessary. A pump may take the form of a mechanically operated syringe.

According to another embodiment of the invention a composition is delivered intra-arterially using protracted infusion. Use of intra-arterial protracted infusion allows the composition to infuse directly into the proliferating structure, via a feeding artery. A catheter may be left in place for several minutes to months in order to deliver the therapy efficiently.

According to one embodiment of the invention, a composition is administered under positive pressure to a proliferating tissue by directed injection into the tissue, for example using a needle and syringe.

According to another embodiment of the present invention, the composition is administered by injection or deposition into a resection cavity, scar or area of proliferating cells. Thus resection cavities and scars after surgical debulking of tumours can be treated. For example, said cavity any of brain tumour resection, breast tumour resection, prostate cancer resection, muscle resection after a sarcoma, uterine laparoscopic myoma resection, head and neck resection cavities, tongue tumour resection, partial upper maxillar resection, liver tumour resection, kidney tumour resection, or bone tumour resection, scar cavity of a melanoma resection. Furthermore, the bed of cheloid scars after resection may be treated by the present composition, in order to avoid cheloid or hypertrophic scar formation in the population known at risk for such reactions.

According to another embodiment of the present invention, said composition is injected into a mass of proliferating cells such as a tumour under visual control, using ultrasound, MRI, CT-scan, PET-CT or any other imaging means. The authors have found that intratumour diffusion is optimal when there is an homogeneous coverage of the whole tumour volume, and that an injection performed tangentially to the tumour volume is less efficacious. Such mode of injection may be performed using a syringe, pump or high pressure as described above.

Administering a composition of the present invention in a proliferating cell mass not only treats the cell mass but also the lymphatic system through which the tumour drains. With conventional therapies, the such as radiotherapy the lymphatic pathways are destroyed. With systemic chemotherapy, there is no selective treatment of the lymphatic pathways. In the present invention, by contrast, both the lymphducts and lymph nodes are selectively treated, in addition to the cellular proliferation.

Slow Release Formulation

According to another embodiment of the invention, a slow release formulation of the composition is delivered by application, injection or puncture to or proximal to the site of proliferation. Preferably it is administered into a mass of proliferating cells. The slow release agent regulates, preferably slows the release of inhibitor from the composition. A single dose can comprise a large or concentrated dose, which, once at the site of proliferation is released at a rate determined by the formulation. This avoids the need for prolonged treatment times associated with and a high the frequency of administration. Another advantage of a slow release formulation is that the composition diffuses day and night, over several days or weeks. The inhibitors can act when a patient is fasting, (e.g. every night) and there is no competition from the degradation products of ingested meals. Furthermore, the present inventors have found that inhibitor uptake can be relatively slow in some tumours by observing ¹⁸F-FDG and ¹¹C-acetate by PET-CT. Although tumours may show as an intense signal due to the sensitivity of the imaging and probe, this is deceptive of the uptake rate which can be relative low e.g in the range of ng/min. Consequently, a slowly releasing inhibitor is better able to match the rate of inhibitor take by the tumour, and avoid wasteful and toxic overdosing.

For instance, a slow release formulation of fluoroacetate may be applied on a cervical area, if a cervical dysplasia is diagnosed. Pathologic areas are usually detected using acetate applied on the cervix; with dysplasic areas indicating a colour change after this application. In the present case, fluoroacetate in a slow release formulation (e.g. polyorthoester polymer) adheres to the cervix and is actively absorbed by the transformed cells. The cells are destroyed, avoiding the need for a superficial laser therapy.

One embodiment of the present invention is a composition as described herein, further comprising one or more slow release agents. Slow release agents may be natural or synthetic polymers, or reabsorbable systems such as magnesium alloys.

Among the synthetic polymers useful according to a slow release formulation of the invention are poly(glycolic) acid, poly(lactic acid) or in general glycolic- and lactic acid based polymers and copolymers. They also include poly caprolactones and in general, poly hydroxyl alkanoates (PHAs) (poly(hydroxy alcanoic acids)=all polyester). They also include Poly (ethylene glycol), poly vinyl alcohol, poly (orthoesters), poly (anhydrides), poly (carbonates), poly amides, poly imides, poly imines, poly (imino carbonates), poly (ethylene imines), polydioxanes, poly oxyethylene (poly ethylene oxide), poly (phosphazenes), poly sulphones, lipids, poly acrylic acids, poly methylmethacrylate (PMMA), poly acryl amides, poly acrylo nitriles (Poly cyano acrylates), poly HEMA, poly urethanes, poly olefins, poly styrene, poly terephthalates, poly ethylenes, poly propylenes, poly ether ketones, poly vinylchlorides, poly fluorides, silicones, poly silicates (bioactive glass). siloxanes (Poly dimethyl siloxanes), hydroxyapatites, lactide-capronolactones and any other synthetic polymer known to a person skilled in the art. A synthetic polymer may also be a hydrogel based on activated polyethyleneglycols combined with alkaline hydrolyzed animal or vegetal proteins.

Among the natural derived polymers useful according to a slow release formulation of the invention, are poly aminoacids (natural and non natural), poly β-aminoesters. They also include poly (peptides) such as: albumines, alginates, cellulose/cellulose acetates, chitin/chitosan, collagene, fibrine/fibrinogen, gelatine, lignine. In general, proteine based polymers. Poly (lysine), poly (glutamate), poly (malonates), poly (hyaluronic acids). Poly nucleic acids, poly saccharides, poly (hydroxyalkanoates), poly isoprenoids, starch based polymers, and any other natural derived polymer known to a person skilled in the art.

Other polymers may be made from hydrogels based on activated polyethyleneglycols (PEGs) combined with alkaline hydrolyzed animal or vegetal proteins.

For both synthetic and natural polymers, the invention includes copolymers thereof are included as well, such as linear, branched, hyperbranched, dendrimers, crosslinked, functionalised (surface, functional groups, hydrophilic/hydrophobic).

The slow release composition may be formulated as liquids or semi-liquids, such as solutions, gels, hydrogels, suspensions, lattices, liposomes; or implants, such as particles, films, rods, fibres, grains. Solid or semi-solid formulations such as rods, fibres or grains improve the ease of administering the implant. Semi-liquid, liquid substance or polymer, or active substance presenting as powder, may be encapsulated in a resorbable capsule or tube made from gelatine, or any polymer degrading rapidly. Any suitable formulation known to the skilled man is within the scope the scope of the invention. According to an aspect of the invention, a composition is formulated such that the quantity of inhibitor is between less than 1% and 60% of total slow-release polymer mass. According to an aspect of the invention, a composition is formulated such that the quantity of inhibitor is between 1% and 50%, 1% and 40%, 1% and 30%, 1% and 20%, 2% and 60%, 5% and 60%,10% and 60%, 20% and 60%, 30% and 60%, or 40% and 60% of total slow-release polymer mass.

Where the slow release agent has the properties to form a semi-solid (gel-like) polymer, the composition may take the form of a foil allowing the release of inhibitor in a controlled fashion, for instance in contact with superficial skin cancers. The foil administers composition directly into the cells owing to the properties of the hydrogel; the high water content of the gel creates a fully communicating structure to the interior of the proliferating mass of cells. Oncotic pressure holds water inside hydrogel and attracts waters from skin, and makes easy transfer of molecules from gel to skin.

One example of such foil may be, for example, a polymer made from hydrogels based on activated polyethyleneglycols (PEGs) combined with alkaline hydrolyzed soya solutions or other animal or vegetal proteins (bovine serum albumin, soya globulin, casein, pea albumin, starch albumine, ovalbumin, etc).

Such a foil could be for instance 3 mm thick and be filled with saline water to a percentage of 90% or more, containing one to several ATP inhibitors.

Such foils may be used easily to treat superficial skin cancers. For instance, squamous cell skin cancers or basal carcinomas present usually with a round shape, for instance, 1, 3, or 5 cm in diameter. When in contact with the superficial skin tumour, such a hydrogel foil hydrates actively the skin, humidifies the epithelium, and allows easy transfer of inhibitor(s) to the superficial tissues of the tumour.

The typical load of such a 3 mm thick hydrogel foil may be in the range of 0.1 mg of TCA inhibitor per square cm of hydrogel or less, and in the range of 1 mg of oxidative phosphorylation inhibitor per square cm of hydrogel or less.

Once in contact with the lesion, the hydrogel releases slowly the inhibitor(s) inside the superficial tumour.

Typically, the major part of each inhibitor will be delivered to the lesion in a period of 4 to 8 hours. The hydrogel may be replaced every day, until the lesion disappears, which should happen within 1 or 2 weeks.

The lesion may be treated in addition using radiotherapy. The standard doses for the treatment (typically 10 times 4 Gy) could be reduced by 20 to 50%.

Solubilising Agents

According to another embodiment of the present invention at least one TCA cycle inhibitor and/or at least one oxidative phosphorylation inhibitor of a composition is coupled to one or more solubilising agents. Such agents change the hydrophilic and hydrophobic profile of the inhibitor, depending on the required solubility. For example, if a composition according to the invention comprises a hydrophilic TCA cycle inhibitor such as fluoroacetate, and a hydrophobic slow release polymer such as polyorthoester, the inhibitor will not adequately solubilise or suspend within the composition. Similarly, a composition according to the invention comprising a very hydrophilic oxidative phosphorylation inhibitor such as rhodamine 123 and polyorthoester slow release agent, will lead to an inadequately solubilised or emulisified composition. Consequently the release properties of the slow release agent may be compromised, and degradation within the body accelerated. To overcome this, the inventors have coupled at least one TCA cycle inhibitor and/or at least one oxidative phosphorylation inhibitor to a solubilising agent which changes the hydrophobicity or hydrophilicity of the inhibitor, depending on the required formulation. The composition so formed is more stable. According to one aspect of the invention, the coupled compound is a prodrug wherein the solubilising agent is cleaved in vivo, so releasing the inhibitor. According to another aspect of the invention, the solubilising agent is cleaved from the inhibitor more rapidly by the proliferating cells.

Cholesterol

According to one aspect of the invention, cholesterol (II) or a derivative thereof is a solubilising agent. One embodiment of the invention is a composition as mentioned herein in which at least one TCA cycle inhibitor and/or at least one oxidative phosphorylation inhibitor is coupled to cholesterol (III) or derivatives thereof: 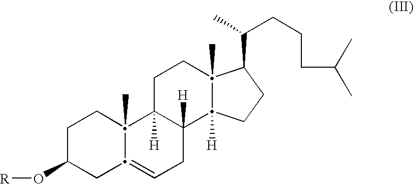
wherein R may be one of the following substances: betahydroxybutyrate, halogenated butyrate, halogenated acetate, halogenated aceto-acetate, halogenated acetamide, halogenated crotonate, halogenated acetone, halogenated citrate and halogenated oleate.

Derivatives of cholesterol are modifications which retain or enhance of activity of the parent compound. Derivatives include, but are not limited to cholesteryl-3-betahydroxybutyrate, cholesteryl-halogenated butyrate, cholesteryl-halogenated acetate, cholesteryl-halogenated aceto-acetate, cholesteryl-halogenated acetamide, cholesteryl-halogenated crotonate, cholesteryl-halogenated acetone, cholesteryl-halogenated citrate, or cholesteryl-halogenated oleate.

Halogenated means fluoro-, chloro-, bromo- or iodo-modified.

An advantage of using cholesterol or a derivative thereof as a solubilising agent is such natural metabolite can enter a cell via a number of mechanisms including through the lipid bilayer of the cell membrane. In rapidly proliferating cells, absorption is more rapid due to the requirement for cholesterol in cell membranes. Once in the lipid bilayer, flippase enzyme transfers the cholesterol-coupled inhibitor from the outer layer to the inner layer; cholesterol is internalised in the cytosol and the inhibitor is released from cholesterol by cholesterol-metabolising enzymes.

A cholesterol is coupled to an inhibitor using known methods. For example, coupling may proceed via nucleophilic attack by electrons of an oxygen atom on the cholesterol. For example, coupling may proceed via nucleophilic attack by electrons of an oxygen atom on the inhibitor. According to another example, esters, ethers or other derivatives of cholesterol or inhibitor may be prepared to facilitate coupling. Mechanisms and knowledge of appropriate coupling moieties are known to the skilled person for the preparation of such coupled inhibitors.

One embodiment of the present invention is a composition comprising cholesteryl-fluoroacetate and polyorthoester.

Vitamin A

According to one aspect of the invention, vitamin A (retinol) or a derivative thereof is a solubilizing agent. One embodiment of the invention is a composition as mentioned herein in which at least one TCA cycle inhibitor and/or at least one oxidative phosphorylation inhibitor is coupled to vitamin A or derivatives thereof. Examples of derivatives include the ether (IV) and ester (V) forms which groups facilitate ease of coupling: 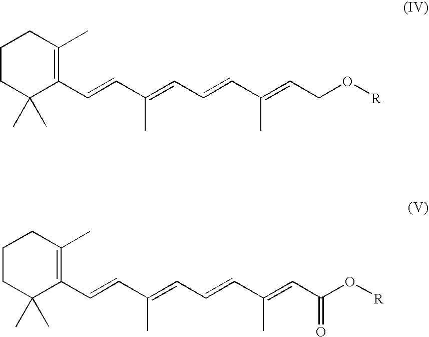
Wherein R may be one of the following substances: betahydroxybutyrate, halogenated butyrate, halogenated acetate, halogenated aceto-acetate, halogenated acetamide, halogenated crotonate, halogenated acetone, halogenated citrate, halogenated oleate.

Derivatives of vitamin A are modifications which retain or enhance of activity of the parent compound. Derivatives include, but are not limited to those mentioned above and betahydroxybutyrate, halogenated butyrate, halogenated acetate, halogenated aceto-acetate, halogenated acetamide, halogenated crotonate, halogenated acetone, halogenated citrate, or halogenated oleate.

Halogenated means fluoro-, chloro-, bromo- or iodo-modified.

An advantage of using vitamin A or a derivative thereof as a solubilising agent is that such natural metabolite can enter a cell via a number of mechanisms. In rapidly proliferating cells, absorption is more rapid, especially in vitamin A metabolising cells such as found in liver tissue. The effect may be used to treat, for instance, hepatocarcinomas by injecting a slow release polymer of retinyl ether or retinoic acids ester coupled with haloacetates directly inside the hepatpcarcinoma mass. The antiproliferative effect commences once the inhibitor is liberated from the polymer and vitamin A is metabolised.

A vitamin A is coupled to an inhibitor using known methods. For example, coupling may proceed via nucleophilic attack by electrons of an oxygen atom on the vitamin A. For example, coupling may proceed via nucleophilic attack by electrons of an oxygen atom on the inhibitor. According to another example, esters or other derivatives of vitamin A or inhibitor may be prepared to facilitate coupling. Mechanisms and knowledge of active groups are known to the skilled person for the preparation of such coupled inhibitors

One embodiment of the present invention is a composition comprising vitamin A-fluoroacetate and polyorthoester.

Encapsulated Inhibitor

According to one aspect of the invention at least one TCA cycle inhibitor and/or at least one oxidative phosphorylation inhibitor of a composition is encapsulated in one or more micro-capsules or nano-capsules.

Examples of nano-capsules (or nano-spheres) or formulations therewith include, but are not limited to a copolymer poly(ethylene oxide) with poly(L-Lactic acid) or with poly(beta-benzyl-L-aspartate); copolymer with poly(lactide-co-glycolide)-[(propylene oxide)-poly(ethylene oxide)]; polyphosphazene derivatives; poly(ethylene glycol) coated nanospheres; poly(isobutylcyanoacrylate) nanocapsules; poly(gamma-benzyl-L-glutamate)/(poly(ethylene oxide); chitosan-poly(ethylene oxide) nanoparticles; nanoparticles where the anti-proliferative drug is prepared using o-carboxymethylate chitosan (o-CMC) as wall forming material; silicone nanocapsules, solid lipid nanospheres or nanoparticles (SLNs) and any known formulation of nano-particles known to someone skilled in the art.

Examples of micro-capsules (or micro-spheres) or formulations therewith include but are not limited to multiporous beads of chitosan; coated alginate microspheres; N-(aminoalkyl) chitosan microspheres; chitosan/calcium alginate beads, poly(adipic anhydride) microspheres; gellan-gum beads; poly(D, L-lactide-co-glycolide) microspheres; alginate-poly-L-lysine microcapsules; crosslinked chitosan microspheres; chitosan/gelatin microspheres; crosslinked chitosan network beads with spacer groups; aliphatic polyesters such as 1,5-diozepan-2-one and D,L-dilactide microspheres; triglyceride lipospheres; polyelectrolyte complexes of sodium alginate chitosan; polypeptide microcapsules; albumin microspheres; and any other micro-capsule (or micro-sphere) formulation known to someone skilled in the art.

By using encapsulated inhibitor, the solubility profile of the inhibitor may be changed according to the environment of the formulation. It may thus act as a solubilising agent as mentioned above. An example of its use is when, an inhibitor of the invention is hydrophilic and a slow-release gel is hydrophobic. An encapsulated inhibitor has an advantage that solubilisation does not require chemical coupling of the inhibitor. Thus, an encapsulated inhibitor allows solubility or emulsification in the slow-release agent, so preventing an otherwise unstable formulation.

Furthermore, encapsulation may be used to modulate the release by the slow-release agent (e.g. fine tune or prolong release time). Furthermore, encapsulation may be used to improve intracellular penetration, as known for encapsulations such as SLN. The advantages of encapsulated formulation may be applied to inhibitor already chemically modified to improve solubility. For example, cholesterol coupled fluoroacetate may be prepared in microcapsules within a slow release gel. The formulation so produced would provide solubility for the inhibitor, slow release modulated by the presence of capsules and active cellular penetration.

Furthermore, encapsulation may be used to modulate release of the inhibitor when a slow-release agent is not present in a composition. For example, when an inhibitor is administered by infusion or injected under high pressure, the composition may comprise one or more TCA cycle inhibitors in the presence of micro- or nano-capsules and/or optionally one or more oxidative phosphorylation inhibitors in the presence of micro- or nano-capsules. Such composition may reduce the frequency and/or duration of treatment compare with conventional formulations.

One embodiment of the present invention is a composition as mentioned above in which at least one inhibitor is encapsulated in micro- or nano-capsule(s) (or micro- or nano-sphere(s)). According to one aspect of the invention, at least one inhibitor is also pre-coupled to a solubilising agent as mentioned above.

Solid Wall Composition

In cases where the composition is viscous, such as, for example, when a particular slow-release agent is present, it can be difficult to administer into a proliferating mass owing to the force required to move the composition through administering tubing or needle. To make administration easier, the composition may be formed into one or more solid wall entities i.e. entities having at least solid or semi-solid walls, which entities are small enough to pass through a needle and into the proliferating mass.

According to one aspect of the invention, a solid wall composition is where the composition is enclosed within a solid or semi-solid bioabsorbable membrane to form a contained capsule. Such capsules are of suitable size and shape (small enough) to pass through a needle and into the proliferating mass. Once administered, the capsule dissolves, and the composition is released. Optionally, the composition may also be disposed on the exterior surface of the capsule, and/or impregnated within the membrane of the capsule. The capsule can be spherical, oval, seed-shaped, tubular or any suitable shape for administration using a needle. The capsule wall can be made of any suitable biocompatible and bioabsorbable material such as, for example, gelatine.

According to another aspect of the invention, a solid wall composition is a solid state bioabsorbable structure, impregnated with composition. Such solid state structures are of suitable size and shape (small enough) to pass through a needle and into the proliferating mass. Once administered, the structure dissolves, and the composition is concomitantly released. The structure can be seed-shaped, rod-shaped or any suitable shape for administration using a needle. The structure can be made of any suitable biocompatible and bioabsorbable material such as, for example, aliphatic polyesters such as homopolymers and copolymers of lactic acid, glycolic acid, lactide, glycolide, para-dioxanone, trimethylene carbonate, epsilon-caprolactone, lactide-capronolactone etc. and blends thereof.

The solid wall composition readily passes through tubing, requiring less force compared with liquid viscous compositions. Furthermore, the precise dose of composition can be administered, and no residual composition is left in the syringe or needle. The solid wall composition can be administered individually, as a series of punctures, for example, one rod per injection. Or, where sufficiently small (nanocapsules), administered in a similar manner to a liquid composition. The maximum width of a solid wall composition is less than the internal diameter of the administering tubing or needle, and can be less than 3 mm, 2 mm, 1 mm, 0.5 mm or a width in the range between any two of the aforementioned widths.

Sensitising Agents

Another embodiment of the present invention is a composition as described herein further comprising one or more components to sensitise the proliferating cells to the inhibitors of the composition. It is achieved by unlocking or unblocking the flow into the TCA cycle. For example, where glycolysis is blocked at the level of pyruvate kinase it is possible to add serine or other elements (e.g. fructose 1-6 diP) to the composition. This forces the pyruvate kinase enzyme to adapt to its tetrameric active form and release the pyruvate kinase inhibition (Mazurek S, Lüftner D, Wechsel H W, Schneider J, Eigenbrodt E. Tumor M2-PK: a marker of the tumor metabolome, in Tumor markers: physiology, pathobiology, technology and clinical applications. Eleftherios P et al, AACC Press 2002, 471-475). The result is a stimulation of the TCA cycle. In cells where the TCA cycle is not very active, this lifts the inhibition, raises the TCA metabolism and, because of that, increase the sensitivity of the cells to TCA inhibitors.

Combined Radiotherapy or Chemotherapy Treatment

Another aspect of the invention, is a method of treating proliferating cells comprising delivering to proliferating cells a composition according to the invention, and radiotherapy and/or chemotherapy. The use of the composition can lead to effective treatment using a fraction of the normal radiotherapy or chemotherapy therapeutic dose.

According to this aspect of the invention, proliferating cells are treated by administering a composition locally, preferably into the proliferating cell mass, as mentioned above. Alternatively, a tumour is totally or partially resected and an implant is placed inside the resection cavity as mentioned above. The site of the proliferation is then treated with radiotherapy applied either from an exterior source, or injection or insertion (manual or automatic) of radioactive isotopes or sources (brachytherapy). The radioactive source may also be a radio-isotope-halogen (RIH) inhibitor as described above. The combination of locally administered composition and radiotherapy treatments may lead to a rapid and effective shrinking or death of the proliferation because it renders the tumour cells much more sensitive.

According to another aspect of the invention, proliferating cells are treated by administering a composition locally, preferably into the proliferating cell mass as mentioned above, and in addition, the site of the proliferation is treated with intravenous chemotherapy (for instance paclitaxel, cisplatinum, vinorelbine, etc). Alternatively, a tumour is totally or partially resected and an implant is placed inside the resection cavity as mentioned above as mentioned above, and in addition, the site of the proliferation is treated with intravenous chemotherapy. The combination of locally administered composition and radiotherapy and/or chemotherapy treatments may lead to a rapid and effective shrinking or death of residual tumour or tumour cells. It is expected that chemotherapy and/or radiotherapy will be much more efficient after the local application of the inhibitors inside the proliferating process. It is foreseen that accumulated doses of radiotherapy and/or chemotherapy could be decreased by 10 to 50%.

One embodiment of the present invention is a method for treating cellular proliferation comprising administering a composition locally, preferably into the proliferating cell mass, as described herein in combination with radiotherapy.

One embodiment of the present invention is a method for treating cellular proliferation comprising administering a composition locally, preferably into the proliferating cell mass, as described herein in combination with chemotherapy.

Another embodiment of the present invention is a method for reducing the dose of radiotherapy treatment of a tumour, comprising administering a composition locally, preferably into the proliferating cell mass, as mentioned above prior to radiotherapy.

Another embodiment of the present invention is a method for reducing the dose of chemotherapy treatment of a tumour, comprising administering a composition locally, preferably into the proliferating cell mass as mentioned above prior to chemotherapy.

Where radiotherapy and chemotherapy are administered, the composition of the present invention may be used to reduce both radiotherapy and chemotherapy doses. A typical chemotherapy and/or radiotherapy dose may be about 20%, 30%, 40%, 50%, 60%, 70%, 80%, 90% less than the dose normally applied to a tumour, in view of the size, location and other factors. It may be a value in the range between any two of the aforementioned values. Preferable, the dose is between 20 and 70% less than the normal dose.

Another embodiment of the present invention is a method for sensitising a proliferating cell mass (e.g. tumour) to radiotherapy, comprising administering a composition locally, preferably into the proliferating cell mass as mentioned above prior to radiotherapy.

Another embodiment of the present invention is a method for sensitising a proliferating cell mass (e.g. tumour) to chemotherapy, comprising administering a composition locally, preferably into the proliferating cell mass as mentioned above prior to chemotherapy.

According to one aspect of the invention, the composition is applied to a subject at least 8 hours, 10 hours, 12 hours, 14 hours, 16 hours, 18 hours, 20 hours, 22 hours, 1 day, 2 days, 3 days, 4 days, 6, days, 8 days, 10 days, 12 days, 14 days, 3 weeks or 4 weeks before the start of radio- and/or chemotherapy, or for a period between any two of the aforementioned periods. Preferably, the compositon is in place for between 12 hours to 4 weeks before commencement of radio- and/or chemotherapy.

Dose

The quantity and concentration of composition for injection and the frequency of administration can be calculated using known techniques by the skilled person.

Typically, 1 to 3-4 grams of the slow release substance may be administered to a 30-50 grams of proliferating cells. The injections may be made in several places inside or proximal to the tumor, in order to warrant an homogeneous distribution of the substances. For instance a 30 grams cervical lymphnode from a tonsillar tumor would receive under ultrasonic control 3 injections of 1 gram of composition in 3 different places of the lymphnode. Each gram of the composition would comprise 0.5 to 2 milligrams of fluoroacetate and 3 milligrams of dinitrophenol, for instance. If taking a tri-block copolymer with a release period of 1 month, the patient is re-examined after 1 month. If the lymphnode did not disappear, but has shrunk to half of its initial size, 1 to 2 injections of 1 gram of the polymer formulation may be repeated 1 month after the first injection, and again 1 month later if necessary to kill all tumor cells.

The active substance may be deposited as a narrow (e.g. 1 to 1.45 mm diameter) cylinder of paste extruded from a needle or tube into the proliferating mass. The polymer will slowly release the product. If several such cylinders are deposited inside a lesion, it ensures a better homogeneity for the therapy. A person skilled in the art will take into account the release rate of the polymer (for instance 5 days or 6 weeks), in order to clinically observe the effects of the treatment. After all the drug has been released, the effect should be considered as being maximal. A repeated therapy will be decided if necessary.

Some proliferating tissues such as cancers are present as huge masses, for instance 5 kgs or 10 kgs. In these cases, the local therapy could be performed when possible as a intra-arterial infusion first, in order to decrease the tumoral volume. When the volume has decreased, after several weeks, the tumor may be implanted with a slow-release polymer containing an active substance in a second step.

According to one aspect of the invention, a composition comprises TCA inhibitor such that the inhibitor concentration delivered to a subject is greater than or equal to 1, 10, 20, 40, 60, 80, 100, 150, 200 mg inhibitor/kg of tumour or of treated mass, or a concentration in the range between any two of the aforementioned values. Preferably the dose is between 1 and 200 mg/kg of tumour or of treated mass.

According to one aspect of the invention, the composition comprising oxidative phosphorylation inhibitor in an amount such that the concentration of inhibitor delivered to a subject is greater than or equal to 5, 10, 15, 20, 25, 30, 35, 40, 45, 50, 55 or 60, 100, 400, 800, 1000, 1500 or 2000 mg inhibitor/kg of tumour or of treated mass, or a concentration in the range between any two of the aforementioned values. Preferably the dose is between 0.010 and 2 g/kg of tumour or of treated mass.

According to one aspect of the invention, a composition comprises an amount of rhodamine to deliver a concentration in the range 0.5 to 3 g/kg, 1 to 2.5 g/kg, and preferably 1.25 to 2.0 g/kg of tumour or of treated mass. According to another aspect of the invention, a composition comprises an amount of rhodamine to deliver a concentration in the range 0.5 to 2 g/kg, 0.75 to 1.75 g/kg, and preferably 1 to 1.5 g/kg of tumour or of treated mass, when used in combination with cytotoxic therapy such as chemo- or radiotherapy.

Imaging Agents

Another embodiment of the present invention is a composition further comprising one or more imaging agents which allow the composition to be viewed using an in vivo imaging device. According to one aspect of the invention, a composition further comprises a magnetic-resonance-visible agent, such as an MR visible polymer, such as a poly(ortho)ester, metallic powder (e.g. tantalum powder), or any other MR visible agent.

According to one aspect of the invention, a composition further comprises a radio-opaque agent, such as, for example, biocompatible metal powder (e.g. tantalum, iridium powder, magnesium alloy powder), or any other agent appear opaque to X-rays. According to another aspect of the invention, a composition further comprises micro-bubbles in order to render the composition ultrasound visible.

The addition of imaging agents allows a physician to accurately administer the composition with the assistance of an in vivo imaging device, and also to follow the progressive polymer erosion inside the tumour mass thereafter.

Combinations

A composition comprising one or more of the aforementioned components is within the scope of the present invention. For example, a composition may comprise one or more TCA cycle inhibitors optionally coupled to solubilising agent, one or more oxidative phosphorylation inhibitors optionally coupled to solubilising agent, one or more slow-release agents, one or more micro- or nano-particles containing at least one of the active substances, one or more one or more sensitising agents, and/or one or more imaging agents, and other components known to the skilled person for suitable formulation of the composition.

Kit

A kit according to the invention comprises at least one composition of the present invention.

It is an aspect of the invention that a composition is provided in a container. For example, a vial, a sachet, a screw-cap bottle, a syringe, a non-resealable vessel, a resealable vessel. Such containers are any that are suitable for containing a composition. Some active products, for example, are sensitive to light and heat and should be preserved in dark and cold.

A kit may provide a range of vials containing different compositions with different inhibitors, different combinations of inhibitors, different combinations of slow-release polymers. A kit may comprise a means for administering the composition (e.g. one or more syringes). A kit may facilitate the sequential application of more than one type of composition. A kit may contain instructions for use.

### EXAMPLES

The invention is illustrated by the following non-limiting examples. They illustrate the effectiveness of a selection of TCA cycle and oxidative phosphorylation inhibitors described above. The inihibitory properties of the other inhibitors not mentioned in the examples are known, and the skilled person may readily substitute the exemplified inhibitors with equivalent pathway inhibitors such as listed above.

### Example 1

A patient has a prostate carcinoma, a PSA level at 10 ng/ml, prostate biopsies showing a gleason score of 7 and lesions present in both lobes. A fluor-choline PET-CT does not show any pathologic lymphnodes in the pelvis. This patient is a candidate for a local therapy such as surgical resection or external or interstitial radiation therapy. Another alternative is to implant the peripheral area of the prostate under ultrasound, MR or CT control with a slow release formulation of fluoroacetate, rhodamine 123 (or dinitrophenol) or a combination of both products. The patient undergoes loco-regional anesthesia and is placed in gynecological position under an open MR machine or using ultrasonic control. A mixture of poly(ortho)ester and fluoroacetate is injected inside the prostate, in the prostatic peripheral area, several mm inside the prostate capsule (5 to 10 mm). 1 to 4 mg of fluoroacetate in total in slow release formulation are injected as 8 peripheral injections. Simultaneously, 6 other injections are performed with 10 mg of dinitrophenol or 30-50 mg rhodamine 123 (or 15 mg rhodamine 6G) between the injection areas of fluoroacetate. As the poly(ortho)esther (POE) is perfectly visualized under MR, one can follow its deposition area, and during following days, the degradation of the POE. The injected POE degrades over 10 to 30 days, depending on the local biological conditions. The fluoroacetate and/or rhodamine are actively absorbed (or by diffusion for dinitrophenol) by the tumor cells. In the following months the therapeutic effect is observed by measuring the PSA blood levels. In case of tumor persistence, a second or a third therapeutic session is realised. The exact tumor area is determined by MR spectroscopy or PET-CT, and this area is implanted exclusively and repeatedly. It is supposed that the injected drug follows the lymphatic pathways and treats microscopic foci in drainage lymphnodes as well.

### Example 2

A female patient presents with a biopsy-proven cervix tumor, 3 cm in diameter. No lymphnodes are seen on the MR or PET-CT examinations. There are several therapeutic alternatives: (a) the patient is operated on, and the lymphnodes are removed (b) the patient receives radiation therapy simultaneously with a cisplatin based chemotherapy, or finally, (c) the patient benefits from one to several injections of fluoroacetate and rhodamine 123 or dinitrophenol in a slow release formulation. Another possibility is to place a tube with holes inside the cervix, and to inject fluoroacetate and dinitrophenol under high pressure from inside the cervical canal, perpendicularly to cervical canal axis, towards the tumor. After several such injections, the tumor shrinks and disappears. The patient is carefully observed, for loco-regional recurrence and for a recurrence in the lymphnodes.

### Example 3

A patient presents with a 4 cm diameter pulmonary mass located in the left inferior lobe. There are bilateral lymphnodes in the mediastinal area and the patient is not considered for a surgical intervention. In order to avoid irradiating the surrounding lung tissue (around the primary tumor), an injection of 1-4 mg of slow release fluoroacetate and 50 mg of rhodamine 123 is performed inside the tumoral mass, under CT guidance. These substances are delivered over the next 10 days. In parallel, a chemoradiotherapy is started on the mediastinal lesions. Some of the mediastinal invaded lymphnodes may be implanted as well, if easily reachable under CT guided puncture. The radiation dose needed to sterilize the injected lesions is possibly reduced from 70-75 to 40-60 Gy.

### Example 4

A patient presents with a cheloid scar. The patient is operated, the cheloid scar is resected, and a catheter is left at the bottom of the scar for several days. After 1-2 weeks, when the sealing process of the scar is underway, a slow release polymer is deposited at the bottom of the resected area using the catheter that was left in place, preventing the cheloid scar formation, which initiates at the bottom of the scar. The amount of fluoroacetate delivered at the bottom of the scar is in the range of several micrograms per cm of scar length. Fluoroacetate may be combined with oxphos inhibitors (0.1 mg per cm of rhodamine 123 or rhodamine 6G for instance).

### Example 5

A 70 years old patient has been operated for a 1 cm breast cancer. The sentinel node technique did not show any invasion. In place of undergoing external beam radiation of the breast, the patient receives an injection of slow release fluoroacetate (1-4 mg) and rhodamine 123 (50 mg), 3 weeks after the surgical excision, inside the surgical scar, under US, CT or MR guidance. This treatment will allow external beam radiotherapy to be avoided, sharply reducing the recurrence rate.

### Example 6

A 33 year old woman presents with a 6 cm diameter myoma, a benign tumor, that grows approximately 2 cm in diameter per year. The standard therapy is the surgical resection of the myoma mass, necessitating a surgical intervention. In the present case, 3 mg of fluoroacetate and 50 mg rhodamine prepared in a slow release formulation are injected twice, 1 month between injections in the myoma mass, leading to its size reduction or disappearance after several months and avoiding the need for a surgical intervention.

### Example 7

A 44 year old patient presents with a 2 cm long esophageal tumor. The echoendoscopic examination shows a 8 mm thick circumferential tumor. A flexible injector is introduced inside the biopsy channel of the endoscope and 4 punctures are performed all around the esophageal circomference, through angulated punctures, inside the tumoral thickening. 1 to 5 mg of fluoroacetate and 30-50 mg of rhodamine 6G in a slow release formulation are delivered. The patient is then submitted to chemotherapy and radiation. Radiation is delivered at a reduced therapeutic dose of 50 Gy instead of 70 Gy and the tumor is totally eradicated.

### Example 8

A 45 y old woman complaints from uterine bleeding. An MRI examination shows 3 benign myomas located in the uterine wall, 5, 3 and 2.5 cm in diameter. The patient undergoes locoregional anesthesia, and under ultrasonic or MRI control all three lesions are punctured and injected with a POE polymer, 1 cc each, each having rhodamine 123, 50 mg per syringe and fluoroacetate embedded in solid-lipid-nanoparticles (SLNs), with 1 mg of fluoroacetate per g of poly(ortho)esther (POE).

### Example 9

A 40 year old patient presents with a malignant tumor in the right tonsillar area, 2 cm in diameter, with a lymphnode of 3 cm of diameter in the right cervical area. 1 g of poly(ortho)esther (POE) is injected in each lesion. The formulation contains 5 mg of dinitrophenol and 1 mg of fluoroaceate attached to cholesterol, mixed to the POE, per gram of POE. The lesions disappear and the patient is regularly followed for recurrence.

### Example 10

A 70 years old patient complains of anal bleeding for several months. A check-up allows to observe a tumour located in the higher part of the rectum ampulla. The tumour is well circumscribed and measures 4 cm in diameter. The patient is implanted with a slow release polymer containing 20 mg of fluorohydroxybutyrate encapsulated in SLN and 3 mg of oligomycin total dose in 4 parallel rods 2 cm long, 2 mm in diameter. After 2 months the patient shows up with a 1.5 cm remaining lesion. A new implant is performed with a single rod in the center of the lesion. 3 months later the tumour has disappeared. The patient is kept under clinical and imaging follow-up.

### Example 11

A 50 y old woman was operated 3 years ago for a melanoma in the left upper part of her back. She is been discovered with a 5 cm right axillar lymphnode. A biopsy confirmed the recurrence of the melanoma. A total dose of 4 mg of fluoroacetate in slow release formulation (to be delivered over 1 week) is injected inside the node in 3 different areas of the lymphnode, under CT scan control. The polymer contains 0.5% of tantalum powder to be visualized under Ctscan. The patient comes back 1 month later with a decrease of the lymphnode to 3 cm. 6 sessions of radiotherapy are performed, 2 sessions a week, at 5 Gy each. The patient is seen 6 months later with a 1 cm remaining in duration in the area of the lymphnode.

### Example 12

A 66 year old woman presents with a bulky right breast tumor, 6 cm in diameter. The tumor is well delineated. The patient refuses standard therapy (surgery, hormone therapy or radiation therapy). She accept a palliative therapy in a pilot trial when a single minimally invasive intervention is performed. Four 3 cm long rods of lactide-capronolactone, containing each 10 mg of sodium arsenate prepared in SLN, are implanted inside the tumour volume under CT scan monitoring. 2 months later the tumor has shrunk to a diameter of 4 cm. A second therapeutic session is performed using 3 rods, with a total dose of 30 mg, and the tumor shrinks again to 3 cm. The patient refuses further treatment and is followed-up for recurrence and new treatment.

### Example 13

A 45 year old patient presents with a 10 cm diameter lymphnode located in the left cervical area. He has been treated 5 years ago for a tonsillar carcinoma by limited surgery and did not show up for further therapy. The patient refuses any kind of prolonged therapy. He accept a tumour implantation of a slow release drug. Fourteen 7 cm long rods of lactide-capronolactone containing rotenone, are implanted. The total dose of rotenone delivered over 2 weeks by the rods is 60 mg. The patient shows up 6 weeks later, and the mass has shrunk to 5.5 cm. The patient accepts radiation therapy. A new implantation is performed with 30 mg of rotenone, and the lymphnode totally disappears at a radiation dose of 50 Gy.

### Example 14

A 46 y old woman presents with metastases originating from a breast tumor operated and irradiated 3 years before. A 3.5 cm metastatis is located in the 10^{th} dorsal vertebra, on the right side of the vertebral body as seen on MR. The patient is young and one wants to keep radiotherapy for further recurrences. She benefits from an intratumoural injection of 1 ml of polymeric paste (poly(orthoesther)), containing 20 mg of Antimycin A formulated as SLN. The paste is deposited in the center of the metastasis under fluoroscopy control. The pain drops on the next day by ⅔. MR scan at 1 and 3 weeks shows the progressive disappearance of the polymer, which is very well identified as a dark spot on MR slices. This allows to visualize the elution rate and the melting process of the polymer, and to better follow and understand the kinetics of the treatment.

At 3 weeks the polymer has nearly totally disappeared, and the patient is sent for the injection of a cement (methylmethacrylate) inside the vertebra, under fluoroscopy. The pain disappears totally and the patient is followed up locally, ready to be treated when new metastases appear.

### Example 15

A 59 y old man presents with a single lung metastasis, 4 cm diameter, originating from a colon cancer operated 2 y before. A 11C-acetate PET-CT is performed which shows high uptake of 11 C-acetate in the metastasis (Standard uptake value or SUV=6), and does not show any other lesion. According to inventor experience, this anticipates an active uptake of Facetate by the tumor. The patient, under general anesthesia, benefits from the deposition of 3 rods of 2 cm of lactide-capronolactone polymer, containing 0.5% of Tantalum powder, 6 mg of fluoroacetate and 30 mg of rhodamine 123, and 30 mg serine total dose. The intervention is performed under Ctscan, in order to be very accurate with the tumor targetting. The lesion diameter diminishes progressively in size down to 1 cm 3 months later. The patent refuses to be operated and is kept under observation.

### Example 16

A 60 y old patient is diagnosed with a 3 cm diameter meningioma in the right retro-orbital area of the brain. A PET CT of the brain is performed using ¹¹C-acetate which confirms high focal ¹¹C-acetate accumulation. 4 mg of fluoroacetate in poly(ortho)ester polymer is injected stereotactically in 2 localisations inside the lesion under CT control. Two months later a new MRI is performed which shows a partial necrosis of the lesion and its reduction to a lesion of 2.5 cm. A surgical intervention is performed. It shows 80% of tumour necrosis on histological examination.

### Example 17

A 40 y old patient presents with a basal carcinoma of the skin, 1 cm in diameter, located in the right side of his thorax. A hydrogel foil, 3 mm thick, 2.5 cm in diameter containing 0.1 mg per square cm of fluoroacetate and 1 mg per square cm of dinitrophenol is deposited on the lesion and fixed with a translucent adhesive bandage. The foil is changed every day during 2 weeks. At 6 weeks the patient is examined, showing only a scar at the place where the lesion was present. The patient is followed up.

1-57. (canceled)

58. A method for the treatment of cellular proliferation comprising administering a composition comprising one or more inhibitors of the citric acid, TCA, cycle, one or more slow release agents and optionally one or more inhibitors of oxidative phosphorylation, wherein said composition is administered into the proliferating cell mass.

59. The method according to claim 58, further comprising treating the proliferating cell mass by radiotherapy, wherein said composition is administered into the proliferating cell mass prior to the radiotherapy.

60. The method according to claim 58, further comprising treating the proliferating cell mass by chemotherapy, wherein said composition is administered into the proliferating cell mass prior to the chemotherapy.

61. The method according to claim 58, wherein said TCA cycle and oxidative phosphorylation inhibitors are administered separately, simultaneously or sequentially.

62. The method according to claim 58, wherein administration leads to inhibition of the TCA cycle and oxidative phosphorylation pathway.

63. The method according to claim 58, wherein said TCA cycle inhibitor is an inhibitor of one or more of pyruvate dehydrogenase, citrate synthase, aconitase, isocitrate lyase, alpha-ketoglutarate dehydrogenase complex, succinyl CoA synthetase, succinate dehydrogenase, fumarase, malate synthase, glutaminase and pyruvate dehydrogenase complex.

64. The method according to claim 58, wherein said TCA cycle inhibitor is fluoroacetate or a pharmaceutically acceptable salt, base, ester or solvate thereof.

65. The method according to claim 58, wherein said TCA cycle inhibitor is fluoroacetate or a stereoisomer, tautomer, racemate, prodrug, metabolite thereof, or a pharmaceutically acceptable salt, base, ester or solvate thereof.

66. The method according to claim 58, wherein said inhibitor of oxidative phosphorylation is an inhibitor of enzyme complex V (F0-F1, ATP synthase).

67. The method according to claim 66, wherein said inhibitor of enzyme complex V is any of dinitrophenol, rhodamine, rhodamine 123, rhodamine 6G or a stereoisomer, tautomer, racemate, prodrug, metabolite thereof, or a pharmaceutically acceptable salt, base, ester or solvate thereof.

68. The method according to claim 58, wherein said inhibitor of oxidative phosphorylation is any of rhodamine, rhodamine 6G, rhodamine 123, dinitrophenol or a stereoisomer, tautomer, racemate, prodrug, metabolite thereof, or a pharmaceutically acceptable salt, base, ester or solvate thereof.

69. The method according to claim 58, wherein the oxidative phosphorylation inhibitor is present in an amount such that the concentration of inhibitor delivered to a subject is between 0.01 and 2 g/kg of tumor of treated mass.

70. The method according to claim 58, wherein said composition further comprises one or more agents to unlock flow into the TCA cycle.

71. The method according to claim 70, wherein said agent to unlock flow into the TCA cycle is serine or fructose 1-6 diP.

72. The method according to claim 58, wherein said composition further comprises one or more imaging agents.

73. The method according to claim 72, wherein said imaging agent is any of poly(ortho)ester, metallic powder, tantalum powder, biocompatible metal powder, magnesium alloy, iridium powder, or micro-bubbles.

74. The method according to claim 58, wherein said slow release agent is any of magnesium alloys, poly(glycolic) acid, poly(lactic acid) or in general glycolic- and lactic acid based polymers, copolymers, poly caprolactones and in general, poly hydroxyl alkanoates, poly(hydroxy alcanoic acids), Poly (ethylene glycol), poly vinyl alcohol, poly (orthoesters), poly (anhydrides), poly (carbonates), poly amides, poly imides, poly imines, poly (imino carbonates), poly (ethylene imines), polydioxanes, poly oxyethylene (poly ethylene oxide), poly (phosphazenes), poly sulphones, lipids, poly acrylic acids, poly methylmethacrylate, poly acryl amides, poly acrylo nitriles (Poly cyano acrylates), poly HEMA, poly urethanes, poly olefins, poly styrene, poly terephthalates, poly ethylenes, poly propylenes, poly ether ketones, poly vinylchlorides, poly fluorides, silicones, poly silicates (bioactive glass), siloxanes (Poly dimethyl siloxanes), hydroxyapatites, lactide-capronolactone, natural and non natural poly amino acids, poly β-aminoesters, albumins, alginates, cellulose/cellulose acetates, chitin/chitosan, collagen, fibrin/fibrinogen, gelatine, lignin, protein based polymers, Poly (lysine), poly (glutamate), poly (malonates), poly (hyaluronic acids), Poly nucleic acids, poly saccharides, poly (hydroxyalkanoates), poly isoprenoids, starch based polymers, copolymers thereof, linear, branched, hyperbranched, dendrimers, crosslinked, functionalised derivatives thereof, hydrogels based on activated polyethyleneglycols combined with alkaline hydrolyzed animal or vegetal proteins.

75. The method according to claim 58, wherein at least one of said inhibitors is coupled to a solubilizing agent.

76. The method according to claim 75, wherein said solubilising agent is cholesterol or a derivative thereof.

77. The method according to claim 76, wherein said cholesterol derivatives are any of cholesteryl-halogenated acetate.

78. The method according to claim 76, wherein solubilising agent is vitamin A or a derivative thereof.

79. The method according to claim 78, wherein the derivative of vitamin A is formula (IV) or (V): 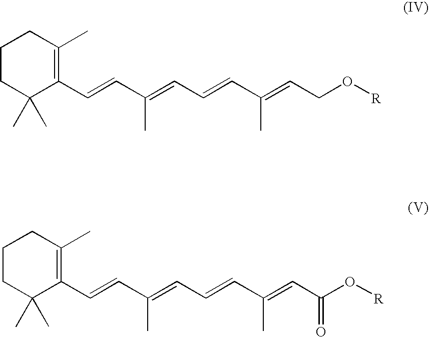
wherein R is selected from the group consisting of halogenated acetate.

80. The method according to claim 58, wherein at least one of said inhibitors is present in a micro-capsule and/or nano-capsule.

81. The method according to claim 80, wherein said nano-capsule is any of copolymer poly(ethylene oxide) with poly(L-Lactic acid) or with poly(beta-benzyl-L-aspartate), copolymer with poly(lactide-co-glycolide)-[(propylene oxide)-poly(ethylene oxide)], polyphosphazene derivatives, poly(ethylene glycol) coated nanospheres, poly(isobutylcyanoacrylate) nanocapsules, poly(gamma-benzyl-L-glutamate)/(poly(ethylene oxide), chitosan-poly(ethylene oxide) nanoparticles, nanoparticles where said inhibitor is prepared using o-carboxymethylate chitosan as wall forming material, or solid lipid nanospheres.

82. The method according to claim 80, wherein said micro-capsule is any of multiporous beads of chitosan, coated alginate micro spheres, N-(aminoalkyl) chitosan micro spheres, chitosan/calcium alginate beads, poly(adipic anhydride) microspheres, gellan-gum beads, poly(D, L-lactide-co-glycolide) microspheres, alginate-poly-L-lysine microcapsules, crosslinked chitosan microspheres, chitosan/gelatin microspheres, crosslinked chitosan network beads with spacer groups, 1,5-diozepan-2-one microspheres, D,L-dilactide microspheres, triglyceride lipospheres, polyelectrolyte complexes of sodium alginate chitosan, polypeptide microcapsules, or albumin microspheres.

83. The method according to claim 58, wherein said composition is administered by infusion into a mass of proliferating cells.

84. The method according to claim 58, wherein said composition is administered by high-pressure injection into a mass of proliferating cells.

85. The method according to claim 58, wherein said composition is administered by direct injection into a mass of proliferating cells.

86. The method according to claim 58, wherein said composition is administered into a resection cavity or scar.

87. The method according to claim 58, wherein said composition is part of a solid wall composition.

88. The method according to claim 87, wherein said solid wall composition is a capsule of suitable size and shape for administration using a needle, said capsule filled with the composition.

89. The method according to claim 88, wherein the wall of said capsule comprises gelatin.

90. The method according to claim 87, wherein said solid wall composition is a solid state bioabsorbable structure of suitable size and shape for administration using a needle, said structure impregnated with the composition.

91. The method according to claim 90, wherein said solid state bioabsorbable structure is seed-shaped, rod-shaped, or tube-shaped.

92. A kit comprising a composition comprising one or more inhibitors of the TCA cycle and/or one or more inhibitors of oxidative phosphorylation.

93. The kit according to claim 92 wherein said TCA cycle inhibitor is an inhibitor of one or more of pyruvate dehydrogenase, citrate synthase, aconitase, isocitrate lyase, alpha-ketoglutarate dehydrogenase complex, succinyl CoA synthetase, succinate dehydrogenase, fumarase, malate synthase, glutaminase and pyruvate dehydrogenase complex and/or said inhibitor of oxidative phosphorylation is an inhibitor of enzyme complex V (F0-F1, ATP synthase).

94. The kit according to claim 92, further comprising a syringe.

95. A hydrogel comprising a) composition comprising one or more inhibitors of the citric acid, TCA, cycle, one or more slow release agents and optionally one or more inhibitors of oxidative phosphorylation, and b) an activated polyethyleneglycol (PEG) combined with any of alkaline hydrolyzed soya solutions, animal or vegetal proteins, bovine serum albumin, soya globulin, casein, pea albumin, starch albumin, or ovalbumin.

96. The hydrogel according to claim 95 wherein the TCA inhibitor of the composition is present at a concentration of less than or equal to 0.1 mg per square cm of hydrogel and/or an oxidative phosphorylation inhibitor of the composition is present at a concentration of less than or equal to 1 mg per square cm of hydrogel.

97. A method of treating superficial cell proliferation comprising applying the hydrogel of claim 95 to the surface of said proliferations, wherein said superficial cell proliferation is basal carcinoma or a squamous cell carcinoma.
